(19) United States
(12) Patent Application Publication
(10) Pub. No.: US 2012/0157379 A1
(43) Pub. Date: Jun. 21, 2012

(54) Gastric Inhibitory Peptide Variants and Their Uses

(76) Inventor: Sheau Yu Hsu, Menlo Park, CA (US)

(21) Appl. No.: 13/387,931

(22) PCT Filed: Jul. 29, 2010
(86) PCT No.: PCT/US10/43749

§ 371 Date: Mar. 2, 2012

Related U.S. Application Data
(60) Provisional application No. 61/273,176, filed on Jul. 31, 2009.

(51) Int. Cl. A61K 38/26; A61P 3/10; C07K 16/18; C12Q 1/68; C07K 14/47; A61K 38/16

(52) U.S. Cl. 514/7.2; 530/324; 514/21.3; 530/387.9; 514/6.9; 435/6.11

(57) ABSTRACT

Novel gastric inhibitory peptide (GIP) polypeptide compositions are provided. Human GIP alleles encode an extended peptide, referred to herein as GIP55S or GIP55G, which is resistant to serum degradation, relative to the known mature GIP peptide. GIP55S or GIP55G peptides find use where it is desirable to modulate insulin secretion.

Diabetes is a metabolic disease that occurs when the pancreas does not produce enough of the hormone insulin to regulate blood sugar (“type 1 diabetes mellitus”) or, alternatively, when the body cannot effectively use the insulin it produces (“type 2 diabetes mellitus”).

According to recent estimates by the World Health Organization, more than 200 million people worldwide have diabetes, whereby 90% suffer from type 2 diabetes mellitus. Typical long term complications include development of neuropathy, retinopathy, nephropathy, generalized degenerative changes in large and small blood vessels and increased susceptibility to infection. Since individuals with type 2 diabetes still have a residual amount of insulin available in contrast to type 1 diabetic individuals, who completely lack the production of insulin, type 2 diabetes only surfaces gradually and is often diagnosed several years after onset, once complications have already arisen.

Insulin resistance occurs in 25% of non-diabetic, non-obese, apparently healthy individuals, and predisposes them to both diabetes and coronary artery disease. Hyperglycemia in type II diabetes is the result of both resistance to insulin in muscle and other key insulin target tissues, and decreased beta cell insulin secretion. Longitudinal studies of individuals with a strong family history of diabetes indicate that the insulin resistance precedes the secretory abnormalities. Prior to developing diabetes these individuals compensate for their insulin resistance by secreting extra insulin. Diabetes results when the compensatory hyperinsulinemia fails. The secretory deficiency of pancreatic beta cells then plays a major role in the severity of the diabetes.

G-protein-coupled receptors (GPCRs) are among the most important pharmaceutical targets, and drugs and peptides modulating GPCR signaling have been widely used in many medical areas including cardiovascular, pulmonary, endocrine, obesity, diabetes, immunology, neuronal diseases, cancer, and infectious diseases.

GPCR signaling plays a vital role in a number of physiological contexts including, but not limited to, metabolism, inflammation, neuronal function, and cardiovascular function. For instance, by way of illustration and not limitation, GPCRs include receptors for biogenic amines, e.g., dopamine, epinephrine, histamine, glutamate, acetylcholine, and serotonin; for purines such as ADP and ATP; for the vitamin niacin; for lipid mediators of inflammation such as prostaglandins, lipoxins, platelet activating factor, and leukotrienes; for peptide hormones such as calcitonin, follicle stimulating hormone, gonadotropin releasing hormone, ghrelin, motilin, neurokinin, and oxytocin; for non-hormone peptides such as beta-endorphin, dynorphin A, Leu-enkephalin, and Met-enkephalin; for the non-peptide hormone melatonin; for polypeptides such as C5a anaphylatoxin and chemokines; for proteases such as thrombin, trypsin, and factor Xa; and for sensory signal mediators, e.g., retinal photopigments and olfactory stimulatory molecules.

GIP secreted from duodenal and the jejunal K-cells is one of the two incretin hormones (glucagon like peptide-1 (GLP-1) and GIP) in humans. It is important for ensuring prompt uptake of glucose and lipids into tissues by stimulating insulin release after food intake. Abnormal regulation of GIP signaling leads to altered carbohydrate metabolism and lipid accumulation, and it was long recognized that diabetes patients have a loss of response to GIP stimulation as compared to healthy individuals. Moreover, it was recently shown that exogenous GIP worsens postprandial hyperglycemia in type II diabetes, and promotes obesity in mice fed a high-fat diet. Thus, the identification of functional GIP variants in humans is relevant to studies of molecular mechanisms underlying diabetes and obesity as well as human adaptations.

There is considerable interest for clinical and research purposes in the discovery and development of agents that act to regulate glucose metabolism, particularly where there can be enhanced activity over existing ligands.

### SUMMARY OF THE INVENTION

Novel gastric inhibitory peptide (GIP) polypeptide compositions are provided. Human GIP alleles encode an extended peptide, referred to herein as GIP55S or GIP55G, which is resistant to serum degradation relative to the known mature GIP peptide. The GIP55G variant exhibits a significantly higher bioactivity as compared to GIP55S. Because GIP and its receptor are crucial to the normal regulation of carbohydrate and lipid metabolism, these peptides are of particular interest for therapeutic methods.

In one embodiment of the invention, GIP55S or GIP55G peptides find use where it is desirable to modulate insulin secretion. Thus, the invention relates to the use of GIP55S or GIP55G and modulators thereof in the treatment of disorders that are benefited from agents useful in modulating insulin secretion, particularly diabetes and more particularly type II diabetes, and hypoglycemia.

In addition to use as a therapeutic agent, in another embodiment of the invention GIP55S or GIP55G peptides are utilized in screening and research methods for the determination of specific analogs, agonists, antagonists mimetics and agents that modulate their production, metabolism, and disposition. The peptides are ligands for G protein-coupled receptors (GPCRs) and can play important roles in the gastrointestinal system.

In one embodiment of the invention, an isolated polypeptide is provided, wherein said polypeptide is encoded by a nucleotide sequence selected from the group consisting of: GIP55S or GIP55G; and functional fragments, derivatives and homologs thereof. Such polypeptides may be formulated in a pharmaceutical composition comprising a pharmaceutically acceptable carrier.

In other embodiments of the invention a method of modulating insulin secretion is provided, wherein a therapeutically effective amount of a GIP55S or GIP55G peptide is administered. Such administering may be performed on an individual suffering from a carbohydrate or lipid metabolic disorder; and the like.

The invention further relates to the use of single nucleotide polymorphisms (SNPs) for identifying an increased risk of type II diabetes and obesity, and to primers, probes and polynucleotides suitable for said use. In addition, the invention relates to the use of GIP for finding active substances for preventing and treating type II diabetes and obesity.

Genetic markers are provided that are useful in determining the predisposition of an individual for development of diabetes mellitus, particularly gestational diabetes mellitus. The methods involve obtaining a genetic sample from an individual, and determining the genotype with respect to the GIP locus. It is shown herein that individuals with a variant allele in the GIP promoter region, i.e. homozygous GIP^{-1920A/A} genotype, have significantly higher serum glucose levels following a challenge, and a significantly higher incidence of glucose intolerance when compared to patients with a homozygous GIP^{-1920G/G} genotype. The genotype may be determined by direct sequence, or by typing with an SNP, including rs3895874, rs3848460, and rs937301.

In one embodiment, as shown in FIGS. 3 and 7, Applicants have provided haplotypes that are useful in differentiating individuals with different capability in the GIP physiology. The data provided herein demonstrates that these haplotypes differ greatly among population, and are important for the interpretation of genome-wide association study of metabolic syndrome. These haplotypes are useful for facilitating personal medicine in diabetes and obesity treatment. For example, people with haplotypes A and H should be accommodated differently because their physiology responses to drug treatment will be different.

In another embodiment, the invention provides for the use of SNPs 36-79 in Table 2 in determining different capability in GIP physiology. These SNPs form the backbone of major haplotype in the region. These SNPs are important components of a subhaplotype group.

Other aspects of the invention and their features and advantages will become apparent from the following description and the appended claims.

### BRIEF DESCRIPTION OF THE DRAWINGS

FIG. 1. Differential distribution of alleles at rs2291725 in HGDP-CEPH (H952 subset) populations. Pie charts represent the proportion of each genotype by the geographic region. The ancestral A-44394131 allele (black pie) has a higher frequency in Africa and America populations, whereas most Eurasia populations have a higher frequency of the derived G-44394131 allele (white pie).

FIG. 2. Significance of population differentiation at rs2291725. a-c) Plots of the degree of LD between each pair of genotyped SNPs in a 200 kb region (44325258-44495998) on chromosome 17 in YRI (a), CEU (b), and CHB+JPT (c). The International HapMap data set was analyzed using the HaploView₃₇. Putative LD block structures (defined by D) are indicated by black triangles. LD structure analysis showed that rs2291725 is not linked with neighboring SNPs in YRI. In CEU and CHB+JPT, rs2291725 is positioned within a 90 kb block with high LD. Red squares represent regions of high degree of LD and high likelihood of odds (LOD) (D′=1, LOD>2). Blue squares represent regions of high LD but with low LOD (D′=1, LOD<2). Blue horizontal bars above LD plots represent gene regions including, CALCOCO2, ATP5G1, UBE2Z, SNF8, GIP, and IGF2BP1. The lower panel visualizes the haplotype structure of the examined region as assessed by HaploView₃₇. Chromosome position of SNPs included in the analysis is shown in Table 1. The position of rs2291725 in exon 4 of GIP in the LD plot is indicated by an arrow. d) Plots of the extent and decay of haplotype homozygosity in the 1.0 Mb region surrounding rs2291725 in YRI, CEU, and CHB+JPT (left panel)₆. These plots are divided into two parts. The upper portion shows haplotypes with the ancestral A-44394131 allele in blue, and the lower portion shows haplotypes with the derived G-44394131 allele in reds. Adjacent haplotypes with the same color carry identical genotypes spanning the region between a select SNP and rs2291725. The haplotype homozygosity extended over 0.5 Mb in multiple haplotypes. Plots of breakdown of EHH over distance between rs2291725 and neighboring SNPs at increasing distances are shown on the right panel. EHH decays much slower at the derived allele (red) as compared to the ancestral allele (blue) in YRI and CHB+JPT. The position of rs2291725 at the center of plots is indicated by an arrow.

FIG. 3. Evolution of haplotypes encompassing ATP5G1, UBE2Z, SNF8, and GIP. The haplotype structure from position 44325258 to 44394253 on chromosome 17 was assessed using HaploView. The distribution of 13 haplotypes (A-M) in YRI, CEU, and CHB+JPT is shown on the lower left panel. Haplotype A that contains the derived G-44394131 allele is indicated by a dotted box. There are 12 haplotypes in YRI, whereas CEU and CHB+JPT are represented by 5 and 4 haplotypes, respectively. An unrooted phylogenetic analysis using the 13 haplotype sequences is shown on the right panel (GeneBee server). The frequency of each haplotype in a select population is indicated by the size of pie at the tip of each branch. Full haplotype sequences are given in FIG. 7.

FIG. 4. Human GIP open reading frame contains multiple basic cleavage sites for the generation of multiple bioactive GIP isoforms. a) Alignment of human GIP (residues Y52 to K107) with corresponding residues of GIP sequences from 18 vertebrates (upper panel). The mature human GIP peptide region is indicated by a dark horizontal bar above the alignment. Putative basic cleavage sites are indicated by a red background. The position of the variable residue 103 is indicated by an arrow. Alternative post-translational processing of proGIP could lead to the generation of a 42-amino-acid mature GIP and extended 55-amino-acid isoforms (GIP55G and GIP55S) that differ at position 52 (lower panel). b) GIP, GIP55G, and GIP55S suppressed exogenous glucose in fasting rats in vivo. Each of the three GIP peptides reduced glucose contents in the blood to basal levels at 1 hr after injection. Each data point represents the mean±SEM of quadruplicate samples. Similar results were observed in five separate experiments.

FIG. 5. Receptor-activation activities of GIP, GIP55G, and GIP55S. a and b) Treatments of GIP receptor-expressing HEK293T cells with GIP, GIP55G, or GIP55S led to dose-dependent increases of cAMP production (a and b, top panels). Cells were treated with synthetic peptides for 12 hr, and the signaling is reported as total cAMP contents in cell lysates. Receptor-activation activities of peptides were also analyzed following incubation with pooled normal human serum (a), or pooled complement preserved human serum (b), for 3, 6, and 12 hr. Error bars represent SEM of triplicate samples. Significant differences in cAMP production between GIP55S or GIP55G55G treatments at a given peptide concentration are indicated by asterisks (P<0.01). In the control group, cells were treated with an aliquot of human serum without a synthetic peptide. Similar results were observed in at least three separate experiments. c) Comparison of the slopes for EC₅₀ trend lines for peptides incubated with pooled human serum for different time-spans. The slope for GIP55G group is significantly different from that of the GIP group (*, significantly different from the GIP group, P=0.0023).

FIG. 6. Fₛₜ values across position 40-50 Mb of human chromosome 17. The Fₛₜ plot consists of pairwise comparisons of CEU-CHB+JPT and YRI-CHB+JPT was generated using Haplotter server₆. The position of rs2291725 is indicated by an arrow. The top 5% cutoffs for Fₛₜ for CEU-CHB+JPT and YRI-CHB+JPT are 0.2055 and 0.3374, respectively.

FIG. 7. Alignments of 13 haplotype block (A-M) sequences found within a 70 kb genomic region neighboring rs2291725 (position 44325258-44394253). Among the 37 SNPs in this region, 22 are linked with rs2291725, and are indicated by asterisks above the alignment. Sequences with an identical nucleotide are highlighted by a select color. The position of individual SNPs are shown on the top, and SNPs that flank the region (rs1962412 and rs2291726) are indicated by arrows. The position of rs2291725 is indicated by a red box. The putative ancestral haplotype from chimpanzee is also shown at the bottom of the alignment.

FIG. 8. Differential distribution of polymorphic alleles at the GIP locus in human populations. A) Visual depiction of genotypes within a 250 kb region around GIP in YRI, CEU, and ASN populations. Polymorphic positions are color coded according to their allelic state. Individuals with a homozygous genotype with ancestral allele and a homozygous genotype with derived allele are represented by blue and yellow dots, respectively. The red dots indicate individuals with a heterozygous genotype. The location of SNPs in the GIP gene region is indicated by a green rectangle box. The position of SNPs within the genomic region is indicated by blue vertical lines at the bottom panel, and rs3895874 at −1920 position of GIP is indicated by a red vertical line. B) Average F_{ST} for genic SNPs at the GIP locus between 11 populations of the HapMap phase III dataset. The diameter of blue circles is proportional to estimate of F_{ST}. Population descriptors: ASW: African ancestry in Southwest USA, CEU: Utah residents with Northern and Western European ancestry from the CEPH collection, CHB: Han-Chinese in Beijing, China, CHD: Chinese in Metropolitan Denver, Colo., GIH: Gujarati Indians in Houston, Tex., JPT: Japanese in Tokyo, Japan, LWK: Luhya in Webuye, Kenya, MEX: Mexican ancestry in Los Angeles, Calif., MKK: Maasai in Kinyawa, Kenya, TSI: Toscans in Italy, YRI: Yoruba in Ibadan, Nigeria.

FIG. 9. Linkage disequilibrium at the GIP locus of HapMap I populations. Left panel: Plots of the degree of linkage disequilibrium (LD) between each pair of genotyped SNPs in a 200 kb region surrounding the GIP locus (chr17:44,295 kb-44,495 kb) in YRI, CEU, and ASN populations. Neighboring genes, including ATP5G1, UBE2Z, and SNF8, are indicated by black bars above LD plots. In CEU and ASN, the 91 kb LD block extended beyond the genic region of GIP, and covered ATP5G1, UBE2Z, and SNF8. In YRI, LD is minimal at the same genomic region. The International HapMap I dataset was analyzed using the HaploView 4.1 with default settings. The color scheme was based on r² values. White areas represent LD with r₂=0. Grey areas represent LD with 0<r₂<1. Black areas represent LD with r₂=1. Right panel: Plots of the degree of LD between 11 pairs of genotyped SNPs in the genic region of GIP (rs12602746 to rs9894411). Red areas represent regions with high degree of LD and high likelihood of odds (LOD) (D′=1, LOD scores>2). Blue areas represent regions with low LOD (D′=1, LOD<2).

FIG. 10. Haplotype mapping of the GIP locus in HapMap I populations. Plots of the haplotype structure of genotyped SNPs in a 200 kb region surrounding GIP in YRI, CEU, and ASN populations. Four SNPs at the 5′ gene region of GIP (rs3895874, rs3809770, rs3848460, and rs937301) are positioned in a haploblock in all three HapMap I populations (61). The position of these SNPs is indicated by red rectangle boxes. The 44 highly linked SNPs in CEU and ASN are indicated by blue rectangle boxes.

FIG. 11. The GIP promoter reporter activity is haplotype-dependent. Upper panel: Schematic representation of luciferase reporters containing three inferred haplotypes (derived: GIP^{-1920AAAA}; ancestral: GIP^{-1920GAGG} and GIP^{-1920GGGG}) found in a 2.15 kb fragment of the GIP promoter. The genomic fragment from position −2073 to +77 by of GIP contains four SNPs (from rs3895874 to rs937301), and three of them are linked. Genomic fragments contain each of the three haplotypes were subcloned in the pGL4.2 luciferase reporter. Lower panel: The luciferase reporter activity in HEK293T cells transfected with combinations of reporter and expression vectors for PAX6 and GATA4. In each well, equal amounts of pCMV and pGL4.2 expression vectors and a one-tenth aliquot of a β-galactosidase expression vector were transfected. The reporter activity is haplotype-dependent in the absence or presence of select transcription factors. Luciferase activities were reported as Relative Light Units (R.L.U) after normalization with β-galactosidase activities in transfected cells. Similar results were obtained in four separate experiments.

### DETAILED DESCRIPTION OF THE INVENTION AND PREFERRED EMBODIMENTS

The regulatory peptides GIP55S or GIP55G are ligands for a subgroup of G protein-coupled receptors (GPCRs) that play important roles in the metabolism of carbohydrates and lipids, particularly in the regulation of insulin secretion. In addition to use as a therapeutic agent, GIP55S or GIP55G peptides are utilized in screening and research methods for the determination of specific analogs, agonists, antagonists and mimetics and inhibitors of their production, metabolism and disposition.

In one aspect, the invention features a method of beneficially regulating insulin secretion in a subject by administering to said subject a therapeutically effective amount of GIP55S or GIP55G.

### GIP Variants

Gastric inhibitory polypeptide (GIP), also known as the glucose-dependent insulinotropic peptide is derived from a 153-amino acid proprotein encoded by the GIP gene and circulates as a biologically active 42-amino acid peptide. GIP is released from the precursor by processing at single arginine residues.

GIP stimulates insulin secretion in the presence of glucose. It is a member of a family of structurally related hormones that includes secretin, glucagon, vasoactive intestinal peptide, and growth hormone-releasing factor. These incretin hormones are defined as intestinal hormones released in response to nutrient ingestion, which potentiate the glucose-induced insulin response. In humans, the incretin effect is mainly caused by glucose-dependent insulin releasing polypeptide GIP, and glucagon-like peptide-1 GLP-1. GIP and GLP-1 are both members of the glucagon peptide superfamily, sharing a close amino acid homology. GIP is secreted by K cells from the upper small intestine while GLP-1 is mainly produced in the enteroendocrine L cells located in the distal intestine. Their effect is mediated through their binding with specific receptors, though part of their biological action may also involve neural modulation.

Circulating levels of GIP and GLP-1 are very low in the fasting state, and rapidly increase following food ingestion. Both GIP and GLP-1 are extensively and rapidly degraded by the enzyme dipeptidyl-peptidase IV (DPP-IV), which cleaves the biologically active forms at the position 2 alanine, resulting in inactive or weak antagonist peptide fragments. The enzyme DPP-IV is widely expressed, including in the vascular endothelium of the capillaries of the villi. These findings suggest that the majority of GIP and GLP-1 arriving in the portal circulation is already inactivated, accounting for their short half life. When administered intravenously in normal subjects and in diabetic patients, the plasma half-life (t_{1/2}) of exogenous GIP is about 5-7 minutes, while the estimated half-life of intact GLP-1 is only about 1-2 minutes.

The effects of GIP and GLP-1 are mediated through their binding with specific receptors. Both GIP and GLP-1 receptors have been cloned. They belong to the 7 transmembrane-domain receptor family coupled to a G-protein. Binding of GIP and GLP-1 peptides with their respective receptor causes an activation of adenylate cyclase via the G protein, and leads to an increase of intracellular cyclic AMP levels. Subsequent activation of protein kinase-A results in a cascade of intracellular events such as increased concentrations of cytosolic Ca²⁺ and, in the case of pancreatic beta cells, enhanced exocytose of insulin-containing granules. Other signalling pathways may also be activated (MAP kinase, Phospho-Inositol-Phosphate PIPS, Protein kinase B pathways).

GIP receptors are expressed in the pancreatic islets, gut, adipose tissue, heart, pituitary, adrenal cortex and in several regions of the brain. GLP-1 receptors are expressed in the gastrointestinal tract, endocrine pancreas (alpha and beta cells), lung, kidneys, heart and in several brain areas (hypothalamus, nucleus of the solitary tract, area postrema).

GIP exerts glucose-dependent stimulatory effects on insulin secretion in animals and humans. Results of studies in humans as well as studies in mice with double knockout of the GIP and GLP-1 receptors consistently showed an additive effect of the two hormones GIP and GLP-1 in the incretin effect.

GIP regulates fat metabolism in adipocytes, including enhanced insulin-stimulated incorporation of fatty acids into triglycerides, stimulation of lipoprotein lipase activity, stimulation of fatty acids synthesis. GIP has been shown to promote beta cell proliferation and cell survival in islet cell line studies. The insulinotropic response to the 42 amino acid GIP administration is defective in diabetic patients.

In some embodiments of the invention, GIP55S or GIP55G are administered in combination with GLP-1 or a GLP-1 analog for the treatment of diabetes. Exenatide is the most advanced candidate drug in the clinical development of GLP-1 analogues. Exenatide is the synthetic version of exendin-4, a peptide originally isolated from the saliva of the lizard Heloderma suspectum (Gila monster), showing a 53% amino acid homology with mammalian GLP-1. Exendin-4 acts as a full agonist at the GLP1 receptor. Liraglutide is a long-acting acylated GLP-1 analogue, acting as a full agonist toward the GLP-1 receptor. CJC-1131 is a GLP-1 analogue with an extended halflife of approximately 10 days in humans. In other embodiments, one or both of GIP55S or GIP55G is administered for the treatment of diabetes.

In other embodiments one or both of GIP55S or GIP55G is administered for the treatment is administered to enhance the metabolism of adipocytes.

The invention provides polypeptides referred to herein as GIP55S or GIP55G, as shown below. The peptides are shown herein to be biologically active, including effects on cells in the gastrointestinal tract.

The GIP propeptide is 57 amino acids in length, which is processed to a mature peptide of 42 amino acids in length. GIP55S and GIP55G are 55 amino acids in length and are active forms without further processing. The forms of GIP include:

| | | length |
|---|---|---|
| Species | Sequence | (aa) |
| GIP55S | YAEGTFISDYSIAMDKIHQQDFVNWLLAQKGKKNDWKHNITQREAR | 55 |
| | ALELASQAN | |
| GIP55G | YAEGTFISDYSIAMDKIHQQDFVNWLLAQKGKKNDWKHNITQREAR | 55 |
| | ALELAQAN | |
| Human | YAEGTFISDYSIAMDKIHQQDFVNWLLAQKGKKNDWKHNITQREAR | 57 |
| proGIP | ALELASQANRK | |
| human | YAEGTFISDYSIAMDKIHQQDFVNWLLAQKGKKNDWKHNITQ | 42 |
| mature GIP | | |
| Chimpanzee | YAEGTFISDYSIAMDKIHQQDFVNWLLAQKGKKNDWKHNITQREAR | 57 |
| | ALELASQANRK | |
| Orangutan | YAEGTFISDYSIAMDKIHQQDFVNWLLAQKGKKNEWKHNITQREAR | 57 |
| | ALELASQANRK | |
| Gorilla | YAEGTFISDYSIAMDKIHQQDFVNWLLAQKGKKNEWKHNITQREAQ | 57 |
| | ALELASQANRK | |
| Rhesus | YAEGTFISDYSIAMDKIHQQDFVNWLLAQKGKKNDWKHNITQREAR | 57 |
| monkey | ALELASQANRK | |
| Dog | YAEGTFISDYSIAMDKIRQQDFVNWLLAQKGKKNDWKHNITQREAG | 57 |
| | ALELAHQSNRK | |
| Cow | YAEGTFISDYSIAMDKIRQQDFVNWLLAQKGKKSDWIHNITQREAG | 57 |
| | ALELAHQSNRK | |
| Cat | YAEGTFISDYSIAMDKIRQQDFVNWLLAQKGKKNEWKHNITQREAG | 57 |
| | ALELAHQSNRK | |
| Pig | YAEGTFISDYSIAMDKIRQQDFVNWLLAQKGKKSEWKHNITQREAR | 57 |
| | ALELAHQSNRK | |
| Horse | YAEGTFISDYSIAMDKIRQQDFVNWLLAQKGKKNDWKHNITQREAR | 57 |
| | ALELTHQSNRK | |
| Rat | YAEGTFISDYSIAMDKIRQQDFVNWLLAQKGKKNDWKHNLIQREAR | 57 |
| | ALELAGQSQRN | |
| Mouse | YAEGTFISDYSIAMDKIRQQDFVNWLLAQRGKKSDWKHNITQREAR | 57 |
| | ALVLAGQSQGK | |
| Guinea pig | YAEGTFISDYSIAMDKIRQQDFVNWLLAQKGKKGDWSHNLTQRDTH | 57 |
| | NPELSRPSQER | |
| Elephant | YAEGTFISDYSIAMDKIRQQDFVNWLLAQKGKKNEWKHNITQREAR | 57 |
| | ALELAVQSNRN | |
| Opossum | YAEGTFISDYSITMDKIMQQDFVNWLLSQKGKKNSWRHNITERKAD | 59 |
| | GTGLTNHSNWNLK | |
| Chicken | YSEATLASDYSRTMDNMLKKNFVEWLLARREKKSDNVIEPYKREAE | 60 |
| | PQLSAVSDQSLDPR | |
| Clawed | YSEAILASDYSRSVDNMLKKNFVDWLLARREKKSENTSEATKREAD | 57 |
| Frog | FQLPDVNMKEK | |
| Zebrafish | YAESTIASDISKIVDSMVQKNFVNFLLNQREKKSEPALTEDPESHI | 53 |
| | FNDLLKK | |

For use in the subject methods, any of the GIP55S or GIP55G forms, including peptides comprising the amino acid sequence set forth above and homologs thereof, modifications thereof, or a combination of forms may be used. Peptides of interest include fragments of at least about 10 contiguous amino acids, more preferably at least about 20 contiguous amino acids, at least about 30 amino acids, at least about 40 amino acids, at least about 50 amino acids, up to the provided peptide sequences, and may extend further to comprise other sequences present in the precursor protein.

The sequence of the GIP55S or GIP55G peptides may be altered from those provided herein in various ways known in the art to generate targeted changes in sequence. The altered peptide will be substantially similar to the sequences provided herein, and preferably will differ by from one to three amino acids. The sequence changes may be suitable substitutions, insertions or deletions. These modified peptides may be used as modulators of activity.

The GIP55S or GIP55G peptides may be joined to a variety of other oligopeptides or proteins for a variety of purposes. Various post-translational modifications may be achieved. For example, by employing the appropriate coding sequences, one may provide farnesylation or prenylation. In this situation, the peptide will be bound to a lipid group at a terminus, so as to be able to be bound to a lipid membrane, such as a liposome.

Modifications of interest that do not alter primary sequence include chemical derivatization of polypeptides, e.g., acetylation, or carboxylation. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids.

The subject peptides may be prepared by in vitro synthesis, using conventional methods as known in the art. Various commercial synthetic apparatuses are available, for example, automated synthesizers by Applied Biosystems, Inc., Foster City, Calif., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus, cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. Preferably, the compositions which are used will comprise at least 20% by weight of the desired product, more preferably at least about 75% by weight, even more preferably at least about 95% by weight, and for therapeutic purposes, preferably at least about 99.5% by weight, in relation to contaminants related to the method of preparation of the product and its purification. The percentages are based upon total protein.

In one preferred embodiment of the invention, the therapeutic peptide consists essentially of a polypeptide sequence set forth herein. By “consisting essentially of” in the context of a polypeptide described herein, it is meant that the polypeptide is composed of a sequence, e.g. a sequence set forth in the seqlist, which sequence may be flanked by one or more amino acid or other residues that do not materially affect any basic characteristics of the polypeptide.

### Pharmaceutical Compositions

The polypeptides described herein and analogs thereof (e.g., pharmaceutically acceptable salts) can serve as the active ingredient in pharmaceutical compositions formulated for the treatment of various disorders as described above. The active ingredient is present in a therapeutically effective amount, i.e., an amount sufficient when administered to substantially modulate the effect of the targeted protein or polypeptide to treat a disease or medical condition mediated thereby.

The compositions can also include various other agents to enhance delivery and efficacy, e.g. to enhance delivery and stability of the active ingredients.

Thus, for example, the compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant.

When the pharmaceutical composition includes a polypeptide as the active ingredient, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lies within a range of circulating concentrations that include the ED₅₀ with little or no toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, or intracranial method.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Suitable formulations for rectal administration include, for example, suppositories, which are composed of the packaged active ingredient with a suppository base. Suitable suppository bases include natural or synthetic triglycerides or paraffin hydrocarbons. In addition, it is also possible to use gelatin rectal capsules, which are composed of a combination of the packaged active ingredient with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are preferably sterile. To the extent that a given compound must be synthesized prior to use, the resulting product is preferably substantially free of any potentially toxic agents, such as any endotoxins, which may be present during the synthesis or purification process. Compositions for parental administration are also preferably sterile, substantially isotonic and made under GMP conditions.

### Uses of GIP55S or GIP55G

In light of the pharmacologic activities of GIP55S or GIP55G, numerous clinical indications are evident. For example, clinical indications for which a GIP55S or GIP55G peptide modulator may find use include the treatment of disorders of carbohydrate or lipid metabolism, including diabetes.

Methods of the invention include administering to an individual having type 2 diabetes, or diagnosed as susceptible to development of type 2 diabetes one or both of GIP55S or GIP55G. Administration may be systemic or localized, where the dosage and timing of administration is determined as described herein.

Determining a therapeutically or prophylactically effective amount of the compositions can be done based on animal data using routine computational methods. In one embodiment, the therapeutically or prophylactically effective amount contains between about 0.01 mg and about 1 g of peptide, etc., as applicable. In another embodiment, the effective amount contains between about 1 mg and about 100 mg of peptide, as applicable. In a further embodiment, the effective amount contains between about 10 mg and about 50 mg of the peptide, as applicable.

Administering the instant compositions can be effected or performed using any of the various methods and delivery systems known to those skilled in the art. The administering can be performed, for example, intravenously, orally, via implant, transmucosally, transdermally, intramuscularly, intrathecally, and subcutaneously. The following delivery systems, which employ a number of routinely used pharmaceutical carriers, are only representative of the many embodiments envisioned for administering the instant compositions.

### Antibodies Specific for GIP55S or GIP55G Polypeptides

The present invention further provides antibodies specific for GIP55S or GIP55G polypeptides, e.g. any one of the variants, polypeptides, or domains described above. Such antibodies are useful, for example, in methods of detecting the presence of GIP55S or GIP55G in a biological sample, and in methods of isolating GIP55S or GIP55G from a biological sample. Antibodies may also be useful as antagonists of GIP55S or GIP55G activity.

The GIP55S or GIP55G polypeptides of the invention are useful for the production of antibodies, where short fragments provide for antibodies specific for the particular polypeptide, and larger fragments or the entire protein allow for the production of antibodies over the surface of the polypeptide. As used herein, the term “antibodies” includes antibodies of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme that generates a detectable product, a green fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

“Antibody specificity”, in the context of antibody-antigen interactions indicates that a given antibody binds to a given antigen, wherein the binding can be inhibited by that antigen or an epitope thereof which is recognized by the antibody, and does not substantially bind to unrelated antigens. Methods of determining specific antibody binding are well known to those skilled in the art, and can be used to determine the specificity of antibodies of the invention for a GIP55S or GIP55G polypeptide, particularly a human GIP55S or GIP55G polypeptide.

Antibodies are prepared in accordance with conventional ways, where the expressed polypeptide or protein is used as an immunogen, by itself or conjugated to known immunogenic carriers, e.g. KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized by cell fusion, and then screened for high affinity antibody binding. The immortalized cells, i.e. hybridomas, producing the desired antibodies may then be expanded. For a more detailed description, see Monoclonal Antibodies: A Laboratory Manual, Harlow and Lane eds., Cold Spring Harbor Laboratories, Cold Spring Harbor, N.Y., 1988. If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage display libraries, usually in conjunction with in vitro affinity maturation.

### Compound Screening

In another aspect, the invention relates to methods for assaying or screening compounds to determine their activities as modulators of the function of the polypeptides described above. Compound screening may be performed using an in vitro model, a genetically altered cell or animal, or purified protein corresponding to any one of the GIP55S or GIP55G forms. One can identify ligands or substrates that bind to, modulate or mimic the action of the peptides, including the identification of modulators.

The polypeptides include those provided herein, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Variants can be designed so as to retain or have enhanced biological activity of a particular region of the protein (e.g., a functional domain where the polypeptide is a member of a protein family, or a region associated with a consensus sequence). Variants also include fragments of the polypeptides disclosed herein, for example, biologically active fragments and/or fragments corresponding to functional domains.

Compound screening identifies modulating agents that modulate function of GIP55S or GIP55G. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like.

The term “modulator” includes any molecule, e.g. protein or pharmaceutical, with the capability of altering or mimicking the physiological function of a GIP55S or GIP55G peptide. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate modulators comprise functional groups necessary for structural interaction with proteins, particularly hydrogen bonding, and typically include at least an amine, carbonyl, hydroxyl or carboxyl group, preferably at least two of the functional chemical groups. The candidate modulator often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate modulators are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example. A number of different types of combinatorial libraries and methods for preparing such libraries have been described, including for example, PCT publications WO 93/06121, WO 95/12608, WO 95/35503, WO 94/08051 and WO 95/30642, each of which is incorporated herein by reference.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Preliminary screens can be conducted by screening for compounds capable of binding to, or interfering in the binding of GIP55S or GIP55G. The binding assays usually involve contacting GIP55S or GIP55G with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots, etc.

The level of expression or activity can be compared to a baseline value. The baseline value can be a value for a control sample or a statistical value that is representative of a control population. Expression or activity levels can also be determined for cells that do not respond to GIP55S or GIP55G as a negative control.

Compounds that are initially identified by any of the foregoing screening methods can be further tested to validate the apparent activity. The basic format of such methods involves administering a lead compound identified during an initial screen to an animal that serves as a model for humans and then determining whether the desired biological function is affected. The animal models utilized in validation studies generally are mammals. Specific examples of suitable animals include, but are not limited to, primates, mice, and rats.

Active test agents identified by the screening methods described herein that modulate GIP55S or GIP55G activity can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, New York).

Once analogs have been prepared, they can be screened using the methods disclosed herein to identify those analogs that exhibit an increased ability to modulate GIP55S or GIP55G activity. Such compounds can then be subjected to further analysis to identify those compounds that appear to have the greatest potential as pharmaceutical agents. Alternatively, analogs shown to have activity through the screening methods can serve as lead compounds in the preparation of still further analogs, which can be screened by the methods described herein. The cycle of screening, synthesizing analogs and re-screening can be repeated multiple times.

### SNP Analysis

Single nucleotide polymorphisms (SNPs) are variants of a particular nucleotide sequence containing substitutions at individual positions and are well known to the skilled worker.

The invention relates to a method for identifying an increased risk of type II diabetes and obesity in an individual, which comprises examining a sample taken from an individual, as to whether either or both alleles of the GIP gene have an SNP of the invention.

Genetic variations may be detected, for example, by direct detection of genetic variations at the chromosomal DNA level by way of molecular-biological analysis of the GIP gene which may contain said genetic variations, here in particular the regions around an SNP of the invention, b) via detection by measuring GIP mRNA expression, c) by indirect detection by way of determining the amounts and/or activity of GIP protein present in cells, tissues or body fluids by means of protein-chemical methods.

Genetic variations or polymorphisms at the nucleic acid level (here chromosomal DNA) in the GIP gene may be detected, for example, by 1) methods based on the sequencing of the nucleic acid sequence of said region of the GIP gene (e.g. pyrosequencing, sequencing using radiolabeled or fluorescent dye-labeled nucleotides or via mass spectrometric analysis of said nucleic acid sequence); 2) methods based on hybridization of nucleic acid sequences of said region of the GIP gene (e.g. by means of “DNA microarrays”); 3) methods based on the analysis of amplification products of the nucleic acid sequence of said region of the GIP gene (e.g. TaqMan analyses).

Genetic variations or polymorphisms at the nucleic acid level (here chromosomal DNA) in the GIP gene at an SNP of the invention may also be detected, for example, on the basis of measuring expressed GIP mRNA via 1) methods based on the hybridization of nucleic acid sequences of the GIP gene (e.g. by means of “DNA microarrays”, Northern blot analyses); 2) methods based on the analysis of amplification products of the nucleic acid sequence of the GIP gene (e.g. “TaqMan” analyses, differential RNA display, representational difference analysis).

In addition, genetic variations or polymorphisms in the GIP gene may be detected via analyzing the amount and/or activity of the GIP protein. The amount and/or activity of the GIP protein may be detected, for example, on the basis of 1) methods based on quantitative detection of the amount of the GIP protein (e.g. Western blot analyses, ELISA test) 2) methods based on functional detection of the activity of the GIP protein via in vitro test systems, for example in human cells, animal cells, bacteria and/or yeast cells.

The detection of genetic polymorphisms of the GIP gene, in particular of the GIP variants described herein may serve, for example, as genetic marker for preventive treatments and preventive measures (medication, lifestyle), (a) in order to delay or even to prevent the onset of diabetes, or to alleviate or stop the severity of the later course and the pathological sequelae, or (b) as genetic marker for adjusting a pharmaceutical dosage or (c) as genetic marker for designing a screening for pharmaceuticals or (d) as genetic marker for identifying and, where appropriate, selecting patients in particular treatments or medical studies.

Accordingly, the present invention also relates to the use of the single nucleotide polymorphism (SNP) of the invention for adjusting the dosage of a pharmaceutical for preventing and/or treating type II diabetes and obesity and also to a method for adapting the dosage of a pharmaceutical for treating and/or preventing type II diabetes and obesity in individuals, which method comprises examining a sample taken from an individual, determining the haplotype, said dosage being adapted as a function of the type of said haplotype.

The present invention describes genetic variants in the GIP gene that are genetic markers for an individual's susceptibility to type 2 diabetes mellitus (T2D). In diagnostic methods of the invention, an individual may be evaluated for the presence of polymorphisms in GIP. In some embodiments the polymorphism is a single nucleotide polymorphism (SNP).

Assessment of risk may also include analysis of genomic regions contiguous with the genetic polymorphisms described herein. Large regions of DNA are often inherited as block and, as such, contain causal as well as non-causal polymorphisms in close proximity. Thus a polymorphism may be inherited as a “linkage disequilibrium block” or “LD block” together with numerous other polymorphisms.

In one embodiment of the invention, diagnosis of a susceptibility to type 2 diabetes is carried out by detecting a predisposing polymorphism in the GIP locus. Specific polymorphisms of interest include those present in an SNP of the GIP locus, e.g. the attached SNP polymorphisms set forth in Table 2, SNPs 36-79. It is shown herein that individuals with a variant allele in the GIP promoter region, i.e. homozygous GIP^{-1920A/A} genotype, have significantly higher serum glucose levels following a challenge, and a significantly higher incidence of glucose intolerance when compared to patients with a homozygous GIP^{-1920G/G} genotype. The genotype may be determined by direct sequence, or by typing with an SNP, including rs3895874, rs3848460, and rs937301.

For determining a susceptibility to type 2 diabetes, hybridization methods, such as Southern analysis, Northern analysis, or in situ hybridizations, can be used (see Current Protocols in Molecular Biology, Ausubel, F. et al., eds, John Wiley & Sons, including all supplements through 1999). For example, a biological sample (a “test sample”) from a test subject (the “test individual”) of genomic DNA, RNA, or cDNA, is obtained from an individual (RNA and cDNA can only be used for exonic markers), such as an individual suspected of having, being susceptible to or predisposed for, or carrying a defect for, type 2 diabetes. The individual can be an adult, child, or fetus. The test sample can be from any source which contains genomic DNA, such as a blood sample, sample of amniotic fluid, sample of cerebrospinal fluid, or tissue sample from skin, muscle, buccal or conjunctival mucosa, placenta, gastrointestinal tract or other organs.

The DNA, RNA, or cDNA sample is then examined to determine which allele of a polymorphism is present in a GIP nucleic acid. The presence of the allele of interest can be indicated by hybridization of the gene in the genomic DNA, RNA, or cDNA to a nucleic acid probe. A “nucleic acid probe”, as used herein, can be a DNA probe or an RNA probe; the nucleic acid probe can contain, for example, at least one polymorphic residue in a GIP nucleic acid. The probe can be any of the nucleic acid molecules described above (e.g., the gene or nucleic acid, a fragment, a vector comprising the gene or nucleic acid, a probe or primer, etc.)

A preferred probe for detecting mRNA or genomic DNA is a labeled nucleic acid probe capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mRNA or genomic DNA, and to distinguish between the risk allele and the protective allele.

The hybridization sample is maintained under conditions that are sufficient to allow specific hybridization of the nucleic acid probe to a GIP nucleic acid. “Specific hybridization”, as used herein, indicates exact hybridization (e.g., with no mismatches). Specific hybridization can be performed under high stringency conditions or moderate stringency conditions, for example, as described above. In a particularly preferred aspect, the hybridization conditions for specific hybridization are high stringency appropriate to the length of the probe.

Specific hybridization, if present, is then detected using standard methods. More than one nucleic acid probe can also be used concurrently in this method. Specific hybridization of any one of the nucleic acid probes to an allele indicated herein as being a predisposing allele is diagnostic for a susceptibility to type 2 diabetes.

In another method of the invention, alteration analysis by restriction digestion can be used to detect an alteration in the gene, if the alteration (mutation) or polymorphism in the gene results in the creation or elimination of a restriction site. A test sample containing genomic DNA is obtained from the individual. Polymerase chain reaction (PCR) can be used to amplify a GIP nucleic acid (and, if necessary, the flanking sequences) in the test sample of genomic DNA from the test individual. RFLP analysis is conducted as described (see Current Protocols in Molecular Biology). The digestion pattern of the relevant DNA fragment indicates the presence or absence of the allele indicated herein as being a predisposing allele in the GIP nucleic acid, and therefore indicates the presence or absence of a susceptibility to type 2 diabetes.

Sequence analysis can also be used to detect specific polymorphisms in a GIP, nucleic acid. A test sample of DNA or RNA is obtained from the test individual. PCR or other appropriate methods can be used to amplify the gene or nucleic acid, and/or its flanking sequences, if desired. The sequence of a GIP nucleic acid, or a fragment of the nucleic acid, or cDNA, or fragment of the cDNA, or mRNA, or fragment of the mRNA, is determined, using standard methods. The sequence of the nucleic acid, nucleic acid fragment, cDNA, cDNA fragment, mRNA, or mRNA fragment is compared with the known nucleic acid sequence of the gene or cDNA or mRNA, as appropriate. The presence of a polymorphism indicated herein as being a predisposing allele in the GIP gene indicates that the individual has a susceptibility to type 2 diabetes.

Allele-specific oligonucleotides can also be used to detect the presence of a polymorphism in a GIP nucleic acid, through the use of dot-blot hybridization of amplified oligonucleotides with allele-specific oligonucleotide (ASO) probes (see, for example, Saiki, R. et al., Nature 324:163-166 (1986)). An “allele-specific oligonucleotide” (also referred to herein as an “allele-specific oligonucleotide probe”) is an oligonucleotide of approximately 10-50 base pairs, preferably approximately 15-30 base pairs, that specifically hybridizes to a GIP nucleic acid, and that contains a polymorphism associated with a susceptibility to type 2 diabetes. An allele-specific oligonucleotide probe that is specific for particular polymorphisms in a GIP nucleic acid can be prepared, using standard methods (see Current Protocols in Molecular Biology). The invention further provides allele-specific oligonucleotides that hybridize to the reference or variant allele of a gene or nucleic acid comprising a polymorphism or to the complement thereof. These oligonucleotides can be probes or primers. With the addition of such analogs as locked nucleic acids (LNAs), the size of primers and probes can be reduced to as few as 8 bases.

A test sample of DNA is obtained from the individual. PCR can be used to amplify all or a fragment of a GIP nucleic acid and its flanking sequences. The DNA containing the amplified GIP nucleic acid (or fragment of the gene or nucleic acid) is dot-blotted, using standard methods (see Current Protocols in Molecular Biology), and the blot is contacted with the respective oligonucleotide probe. The presence of specific hybridization of the probe to the amplified GIP nucleic acid is then detected. Hybridization of an allele-specific oligonucleotide probe to DNA from the individual is indicative of the presence of an allele indicated herein as being a predisposing allele in the GIP nucleic acid, and is therefore indicative of susceptibility to type 2 diabetes.

An allele-specific primer hybridizes to a site on target DNA overlapping a polymorphism and only primes amplification of an allelic form to which the primer exhibits perfect complementarity. See Gibbs, Nucleic Acid Res. 17, 2427-2448 (1989). This primer is used in conjunction with a second primer, which hybridizes at a distal site. Amplification proceeds from the two primers, resulting in a detectable product, which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

In another aspect, arrays of oligonucleotide probes that are complementary to target nucleic acid sequence segments from an individual can be used to identify the presence of polymorphic alleles in a GIP nucleic acid. For example, in one aspect, an oligonucleotide array can be used. Oligonucleotide arrays typically comprise a plurality of different oligonucleotide probes that are coupled to a surface of a substrate in different known locations. These oligonucleotide arrays, also described as “Genechips™,” have been generally described in the art, for example, U.S. Pat. No. 5,143,854 and PCT patent publication Nos. WO 90/15070 and 92/10092. These arrays can generally be produced using mechanical synthesis methods or light directed synthesis methods that incorporate a combination of photolithographic methods and solid phase oligonucleotide synthesis methods. See Fodor et al., Science 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186, the entire teachings are incorporated by reference herein. Techniques for the synthesis of these arrays using mechanical synthesis methods are described in, e.g., U.S. Pat. No. 5,384,261; the entire teachings are incorporated by reference herein. In another example, linear arrays can be utilized.

Once an oligonucleotide array is prepared, a nucleic acid of interest is hybridized with the array and scanned for polymorphisms. Hybridization and scanning are generally carried out by methods described herein and also in, e.g., published PCT Application Nos. WO 92/10092 and WO 95/11995, and U.S. Pat. No. 5,424,186, the entire teachings are incorporated by reference herein. In brief, a target nucleic acid sequence that includes one or more previously identified polymorphic markers is amplified by well-known amplification techniques, e.g., PCR. Typically, this involves the use of primer sequences that are complementary to the two strands of the target sequence both upstream and downstream from the polymorphism. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Additional uses of oligonucleotide arrays for polymorphism detection can be found, for example, in U.S. Pat. Nos. 5,858,659 and 5,837,832, the entire teachings of which are incorporated by reference herein. Other methods of nucleic acid analysis can be used to detect polymorphic alleles in a type 2 diabetes gene or variants encoded by a type 2 diabetes gene. Representative methods include direct manual sequencing (Church and Gilbert, Proc. Natl. Acad. Sci. USA 81:1991-1995 (1988); Sanger, F. et al., Proc. Natl. Acad. Sci. USA 74:5463-5467 (1977); Beavis et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V. C. et al., Proc. Natl. Acad. Sci. USA 86:232-236 (1989)), mobility shift analysis (Orita, M. et al., Proc. Natl. Acad. Sci. USA 86:2766-2770 (1989)), restriction enzyme analysis (Flavell et al., Cell 15:25 (1978); Geever, et al., Proc. Natl. Acad. Sci. USA 78:5081 (1981)); heteroduplex analysis; chemical mismatch cleavage (CMC) (Cotton et al., Proc. Natl. Acad. Sci. USA 85:4397-4401 (1985)); RNase protection assays (Myers, R. M. et al., Science 230:1242 (1985)); use of polypeptides which recognize nucleotide mismatches, such as E. coli mutS protein; allele-specific PCR, for example.

In one aspect of the invention, diagnosis of a susceptibility to type 2 diabetes can also be made by expression analysis by quantitative PCR (kinetic thermal cycling). This technique, utilizing TaqMan® assays, can assess the presence of an alteration in the expression or composition of a GIP nucleic acid or splicing variants encoded by a GIP nucleic acid. TaqMan® probes can also be used to allow the identification of polymorphisms and whether a patient is homozygous or heterozygous. Further, the expression of the variants can be quantified as physically or functionally different.

In another aspect of the invention, diagnosis of a susceptibility to type 2 diabetes can be made by examining expression and/or composition of a GIP polypeptide, by a variety of methods, including enzyme linked immunosorbent assays (ELISAs), Western blots, immunoprecipitations and immunofluorescence. A test sample from an individual is assessed for the presence of an alteration in the expression and/or an alteration in composition of the polypeptide encoded by a GIP nucleic acid, or for the presence of a particular variant encoded by a GIP nucleic acid. An alteration in expression of a polypeptide encoded by a GIP, SPP1 or HDC nucleic acid can be, for example, an alteration in the quantitative polypeptide expression (i.e., the amount of polypeptide produced); an alteration in the composition of a polypeptide encoded by a GIP nucleic acid is an alteration in the qualitative polypeptide expression (e.g., expression of an altered GIP polypeptide or of a different splicing variant). In a preferred aspect, diagnosis of a susceptibility to type 2 diabetes can be made by detecting a particular splicing variant encoded by that GIP nucleic acid, or a particular pattern of splicing variants.

Both such alterations (quantitative and qualitative) can also be present. The term “alteration” in the polypeptide expression or composition, as used herein, refers to an alteration in expression or composition in a test sample, as compared with the expression or composition of polypeptide by a GIP in a control sample. A control sample is a sample that corresponds to the test sample (e.g., is from the same type of cells), and is from an individual who is not affected by a susceptibility to type II diabetes. An alteration in the expression or composition of the polypeptide in the test sample, as compared with the control sample, is indicative of a susceptibility to type 2 diabetes. Similarly, the presence of one or more different splicing variants in the test sample, or the presence of significantly different amounts of different splicing variants in the test sample, as compared with the control sample, is indicative of a susceptibility to type 2 diabetes.

Various means of examining expression or composition of the polypeptide encoded by a GIP nucleic acid can be used, including: spectroscopy, colorimetry, electrophoresis, isoelectric focusing, and immunoassays (e.g., David et al., U.S. Pat. No. 4,376,110) such as immunoblotting (see also Current Protocols in Molecular Biology). For example, in one aspect, an antibody capable of binding to the polypeptide (e.g., as described above), preferably an antibody with a detectable label, can be used. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling a DNA probe with biotin such that it can be detected with fluorescently labeled streptavidin.

Western blotting analysis, using an antibody that specifically binds to a polypeptide encoded by an altered GIP nucleic acid or an antibody that specifically binds to a polypeptide encoded by a non-altered nucleic acid, or an antibody that specifically binds to a particular splicing variant encoded by a nucleic acid, can be used to identify the presence in a test sample of a particular splicing variant or of a polypeptide encoded by a polymorphic or altered GIP nucleic acid, or the absence in a test sample of a particular splicing variant or of a polypeptide encoded by a non-polymorphic or non-altered nucleic acid. The presence of a polypeptide encoded by a polymorphic or altered nucleic acid, or the absence of a polypeptide encoded by a non-polymorphic or non-altered nucleic acid, is diagnostic for a susceptibility to type 2 diabetes, as is the presence (or absence) of particular splicing variants encoded by the GIP nucleic acid.

In one aspect of this method, the level or amount of polypeptide encoded by a GIP nucleic acid in a test sample is compared with the level or amount of the polypeptide encoded by the GIP in a control sample. A level or amount of the polypeptide in the test sample that is higher or lower than the level or amount of the polypeptide in the control sample, such that the difference is statistically significant, is indicative of an alteration in the expression of the polypeptide encoded by the GIP nucleic acid, and is diagnostic for a susceptibility to type 2 diabetes. Alternatively, the composition of the polypeptide encoded by a GIP nucleic acid in a test sample is compared with the composition of the polypeptide encoded by the GIP nucleic acid in a control sample (e.g., the presence of different splicing variants). A difference in the composition of the polypeptide in the test sample, as compared with the composition of the polypeptide in the control sample, is diagnostic for a susceptibility to type 2 diabetes. In another aspect, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of a susceptibility to type 2 diabetes.

### Experimental

The following examples are put forth for illustrative purposes, and are not intended to limit the scope of what the inventors regard as their invention. Unless indicated otherwise, parts are parts by weight, molecular weight is weight average molecular weight, temperature is in degrees Celsius, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. Accordingly, it should be understood that the scope of the invention is not limited by this detailed description, but by the appended claims as properly construed under principles of patent law.

### EXAMPLE 1

### Selection of a Cryptic Incretin Hormone in Human Populations

The spread of humans on major continents was accompanied by adaptation to natural environments and changing cultures. Recently, analyses of single nucleotide polymorphisms (SNPs) have revealed genetic loci associated with human adaptations, such as skin and eye color, density of hair follicles, degree of lactose tolerance, and immune responses, as well as genetic predisposition to various diseases. Here, we report that a nonsynonymous SNP, rs2291725, in the glucose-dependent insulinotropic polypeptide (or gastric inhibitory peptide, GIP), was differentially selected in world populations. Comparative and functional analyses showed that the human GIP gene encodes an extended peptide (GIP55S or GIP55G) which encompasses rs2291725 and is resistant to serum degradation relative to the known mature GIP peptide. Importantly, we found the GIP variant encoded by the derived allele (GIP55G) exhibits a significantly higher bioactivity as compared to GIP55S which is derived from the ancestral allele. Because GIP and its receptor are crucial to the normal regulation of carbohydrate and lipid metabolism, our study indicates that geographical variations in allele frequency of rs2291725 could be associated with human adaptation to changing diets and cultures, and sheds light on how our endocrine system evolved as part of the agricultural domestication process.

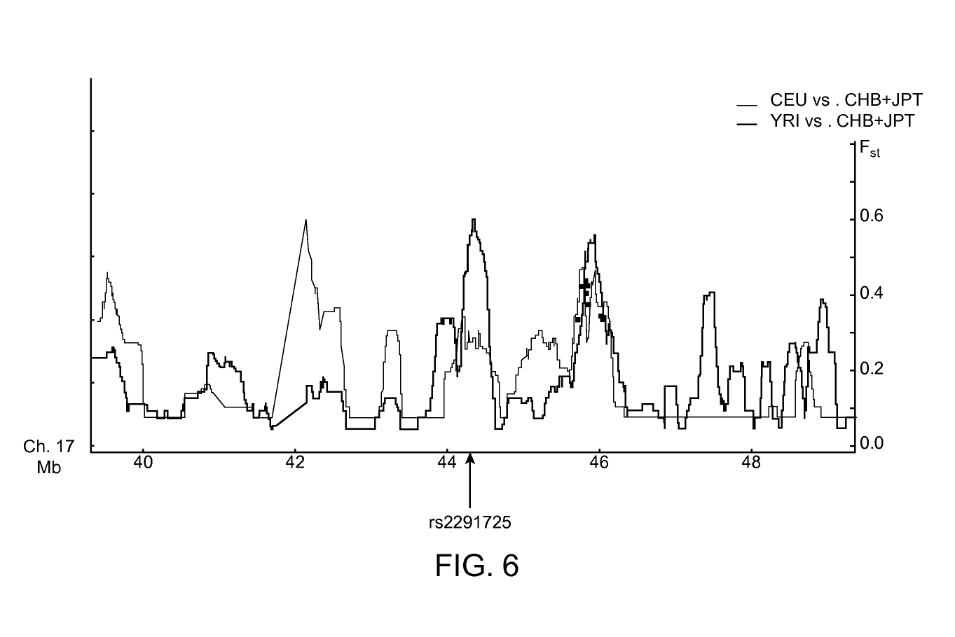

Human SNPs that are distinguished by high population differentiation are regarded as signatures of natural selection in different geographical regions after migration out of Africa. Recent studies have shown that genes associated with intercellular communication, or responses to environmental factors (e.g., pathogens and food sources), are particularly susceptible to selection pressure. Among these genes, G protein-coupled receptors (GPCR, the largest membrane-anchored receptor family) and their ligands have been implicated in the recent evolution of common traits and a variety of pathologies. To examine the extent of contribution of GPCR ligand variation in human adaptation and identify causative signaling hormones, we screened SNPs in the gene region of 117 polypeptide hormones that signal through GPCRs for signals of such selection using the Wright's fixation index F_{ST} (a measure of the degree of differentiation between subpopulations). Analysis of the International HapMap data revealed that a nonsynonymous SNP in exon 4 of GIP (Table 1; rs2291725; nucleotide 44394131, A/G; AGT→GGT, S103G) on chromosome 17q21.3-q22 has an F_{ST} value in the top 2 percentile in comparisons between East Asia (CHB+JPT, Chinese from Beijing and Japanese from Tokyo) and Yoruba (YRI, Yoruba from Ibadan) populations (FIG. 6; Table 2).

Furthermore, analysis of the neighboring genomic region showed that more than 20 SNPs within the adjacent 100 kb of rs2291725 have comparable allele frequencies and F_{ST} values (Table 2). Whereas the average frequency of the ancestral (A-44394131) and the derived (G-44394131) alleles at rs2291725 is approximately even in the overall HapMap population, ˜95% of chromosomes from YRI contain the ancestral allele. In contrast, frequencies of the ancestral allele in CEU (51.7%, U.S. residents with northern and western European ancestry) and CHB+JPT (24.5%) are significantly lower than that of YRI (Table 1; P<0.01).

GIP encodes one of the three major incretin hormones (GIP, glucagon like peptide-1 (GLP-1), and amylin) in mammals, and is essential for normal carbohydrate and lipid metabolism. After ingestion of nutrients, GIP secreted from duodenal and the jejunal K-cells acts on pancreatic β-cells to stimulate the release of insulin, thereby ensuring prompt uptake of glucose and lipids into tissues. Abnormal regulation of GIP signaling leads to the development of type II diabetes and obesity. In clinics, exogenous GIP has been used to treat diabetes26, and the ablation of GIP signaling has been considered useful for the treatment of obesity. Thus, the observed high population differentiation at rs2291725 and the neighboring SNPs could be associated with recent adaptations of humans to changes in diets and cultures. To test this hypothesis, we fine mapped the GIP locus and tested function of the variants in vitro and in vivo.

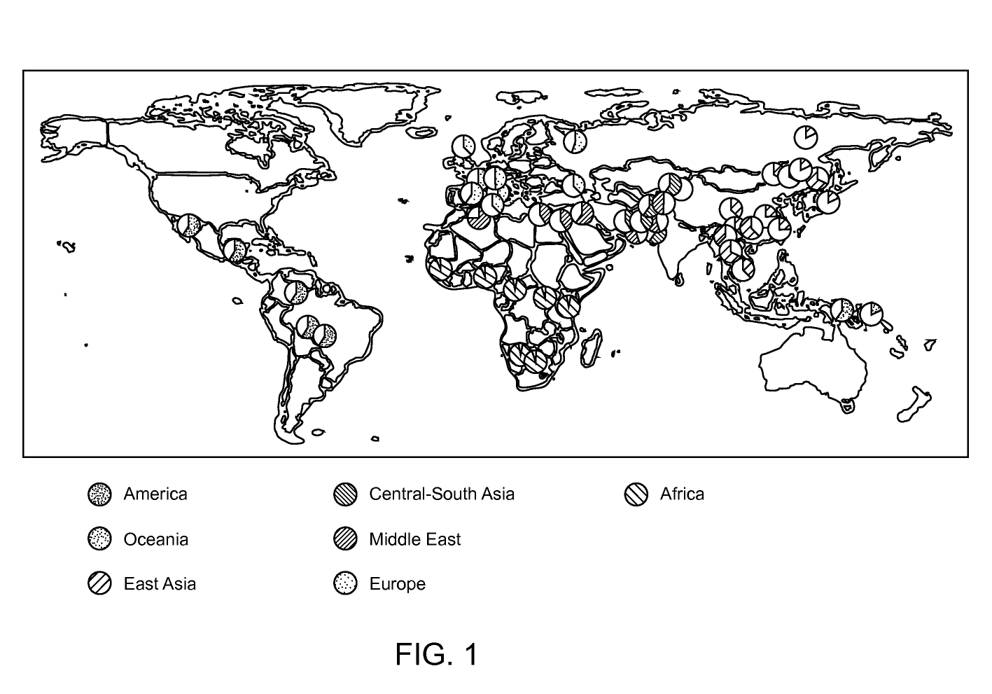

Consistent with findings from the HapMap data, the analysis of genotyping data from the HGDP-CEPH project (Centre d'Etude du Polymorphisme Humain-Human Genome Diversity Panel; 944 individuals from 52 populations) and 752 Japanese individuals (A-44394131=0.268, G-44394131=0.732) from the Human Genome Center at the University of Tokyo confirmed that the allele frequency at rs2291725 varies significantly among world populations_{21,28-31} (FIG. 1, Table 3). Frequency of the derived G-44394131 allele in Sub-Saharan Africans ranges from 0-3.8% in hunter-gatherer populations such as Biaka pygmy, Mbuti pygmy, and San, to 6.3-9.5% in South and West Africa populations. In contrast, the frequency of G-44394131 allele is greater than 40% in Europe and Middle East populations, and increases to >65% in most East Asia populations. In addition, the analysis showed that populations with a shared ancestry, notably, those from Africa, Middle East and Europe, South/Central Asia, and East Asia, share a similar distribution of allele frequency.

However, the allele frequency did not show a strict correlation with the geographic distance to our African source, South-western Africa, suggesting that geographic proximity is not a strong predictor of the rs2291725 allele frequency. The frequency of the derived G-44394131 allele in several South America populations who practiced a hunter-gatherer culture in the forest jungle (Karitiana, Surui, Piapoco and Curripaco) is approximately 20-25%.

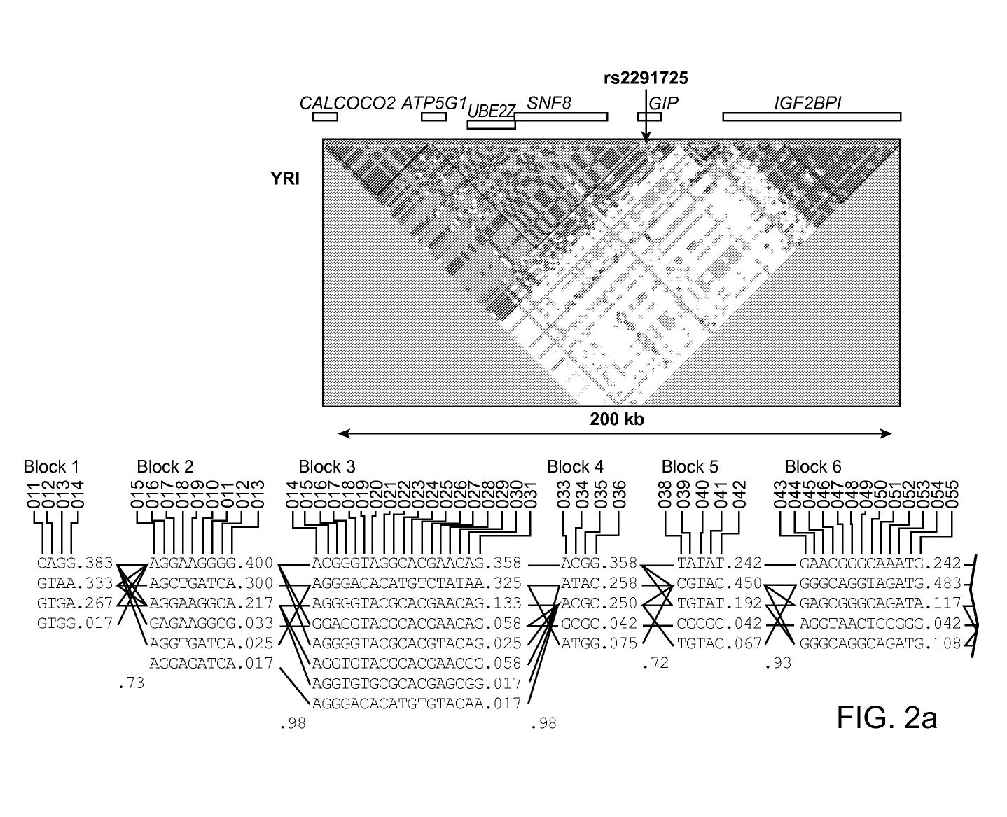

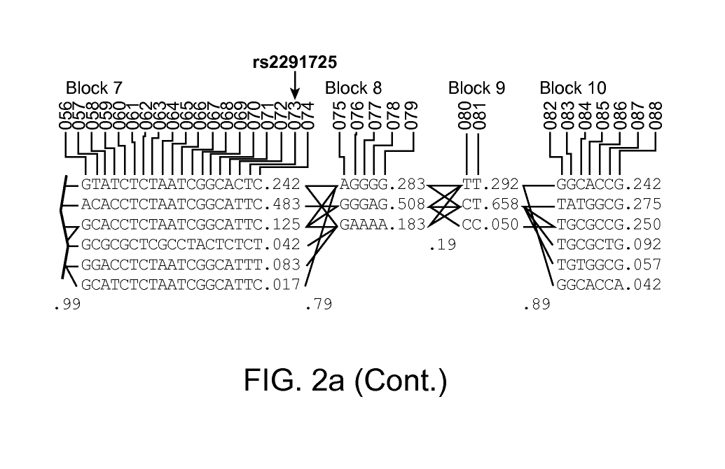

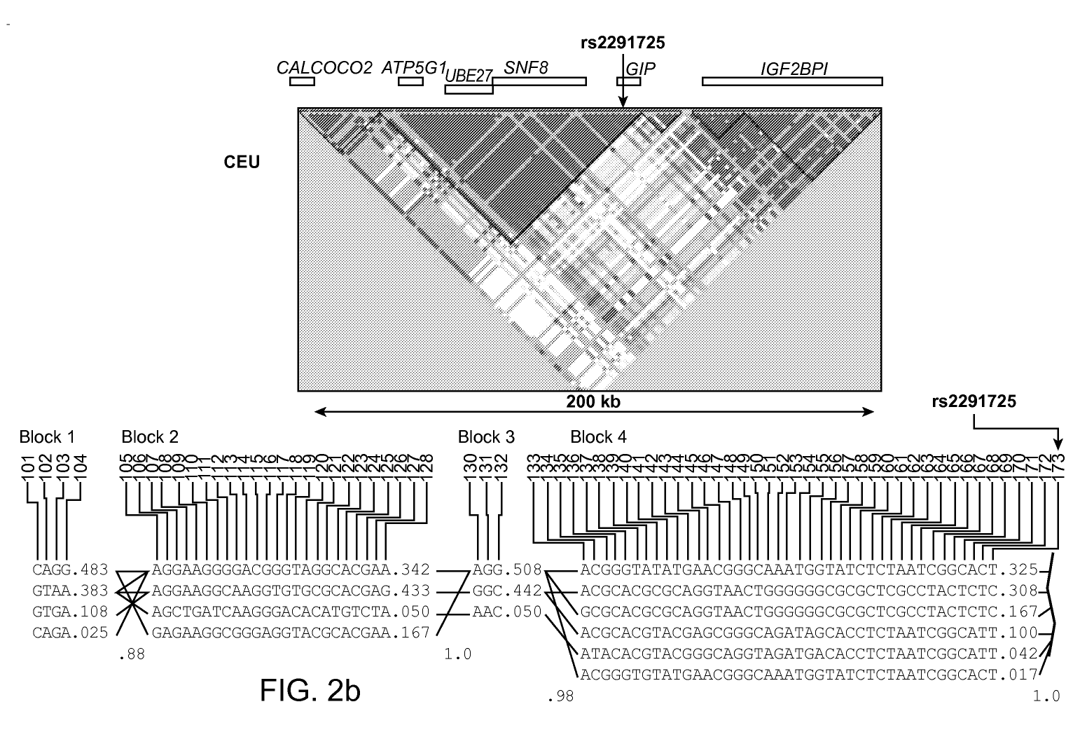

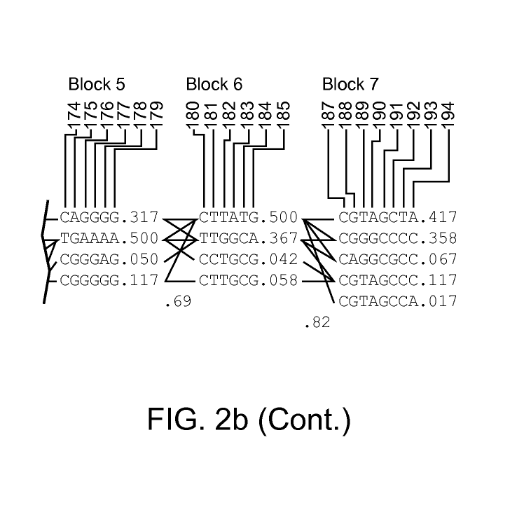

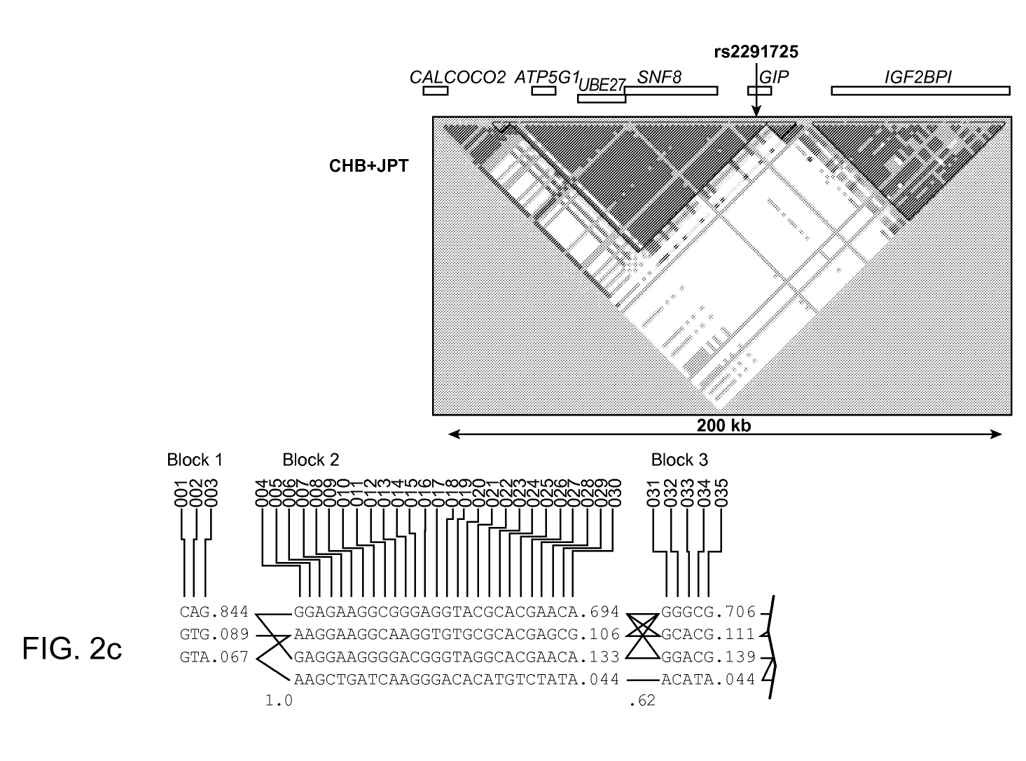

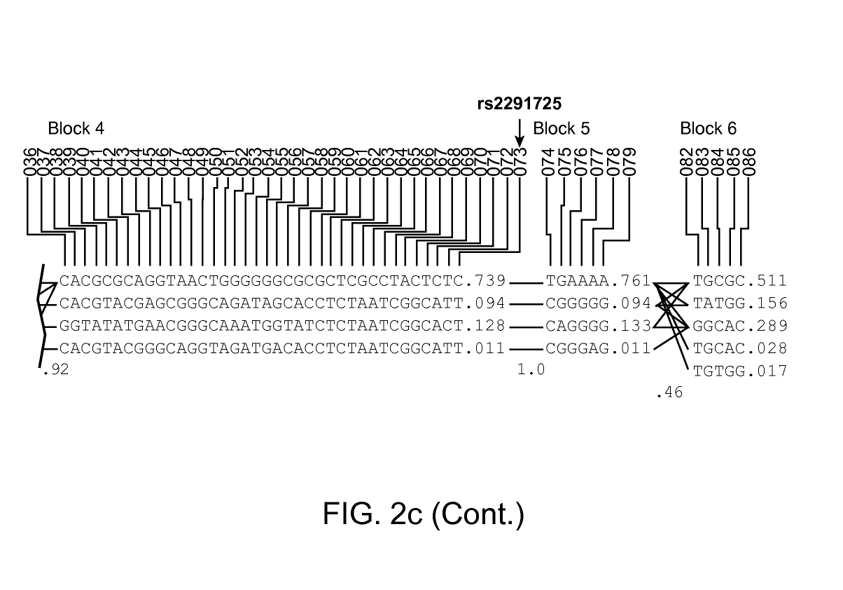

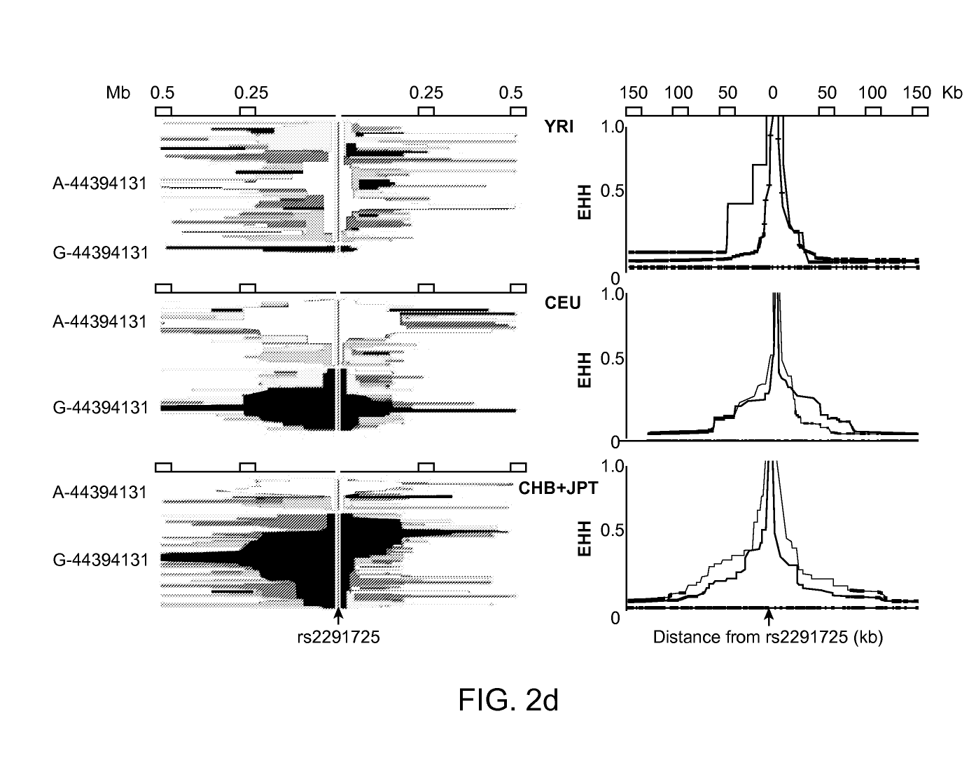

To evaluate and visually assess whether the high F_{ST} at rs2291725 and neighboring SNPs arose from positive selection35, we analyzed patterns of linkage disequilibrium (LD), the extended haplotype homozygosity (EHH), and the decay of haplotype homozygosity. LD plot analysis indicates the presence of a LD block of approximately 80 kb that encompasses the neighboring SNF8 (ESCRT-II complex subunit SNF8) and UBE2Z(ubiquitin-conjugating enzyme E2Z), but not the GIP in YRI (FIG. 2a, upper panel). Linkages in this LD block and the neighboring GIP locus have low likelihood of odds (LOD) and low r² (FIG. 2a, blue square, D′=1, LOD scores<2). In addition, the analysis showed that extensive recombination in this region has resulted in strong haplotype diversity in YRI (FIG. 2a, lower panel). In contrast, an extended LD block of approximately 90 kb with high LD and high r₂ (red square, D′=1, LOD scores>2) is present in the populations of CEU and CHB+JPT, and these LD blocks encompass ATP5G1, SNF8, UBE2Z, and GIP (FIGS. 2b and 2c, upper panel). The haplotype block diversity in the CEU and CHB+JPT populations is significantly lower than that of YRI (FIGS. 2b and 2c, lower panel; CHITEST P=0.047). These data thus suggested that rs2291725 and a number of neighboring SNPs are linked and rose to high frequency in select populations due to genetic hitchhiking36, and the modes of selection at the GIP locus were different among populations37.

Consistent with the LD analysis, plots depicting EHH showed that most of the chromosomes with the derived G-44394131 allele in the CHB+JPT population have EHH extending over 200 kb, and exhibit a significantly longer haplotype as compared to chromosomes with the ancestral A-44394131 allele (FIG. 2d, left bottom panel, P<0.001). In contrast, the YRI chromosomes with the high frequency ancestral A-44394131 allele showed minimal EHH (FIG. 2d, left top panel). However, chromosomes with the low frequency G-44394131 allele in YRI exhibited a long stretch of homozygosity, similar to that of CHB+JPT. On the other hand, in the population of CEU the two variants exhibited a similar extent of EHH (FIG. 2d, left middle panel). These results were reflected in the plots of EHH decay (FIG. 2d, right panel). In the populations of CHB+JPT and YRI, the breakdown of EHH between rs2291725 and the neighboring SNPs was much slower in chromosomes carrying G-44394131 as compared to those carrying the alternative allele. In contrast, the two alleles showed a similar decay pattern in the CEU population. The directional selection of alleles at rs2291725 and neighboring SNPs in these populations is further supported by the analysis of integrated haplotype score (iHS), and Fay and Wu's H statistics (Table 1).

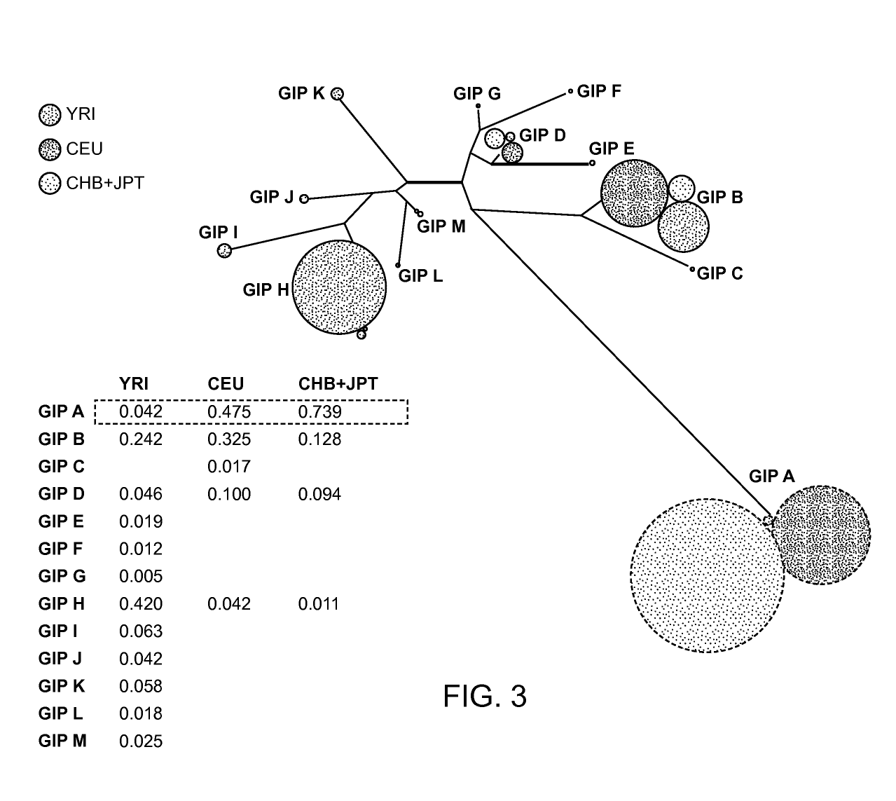

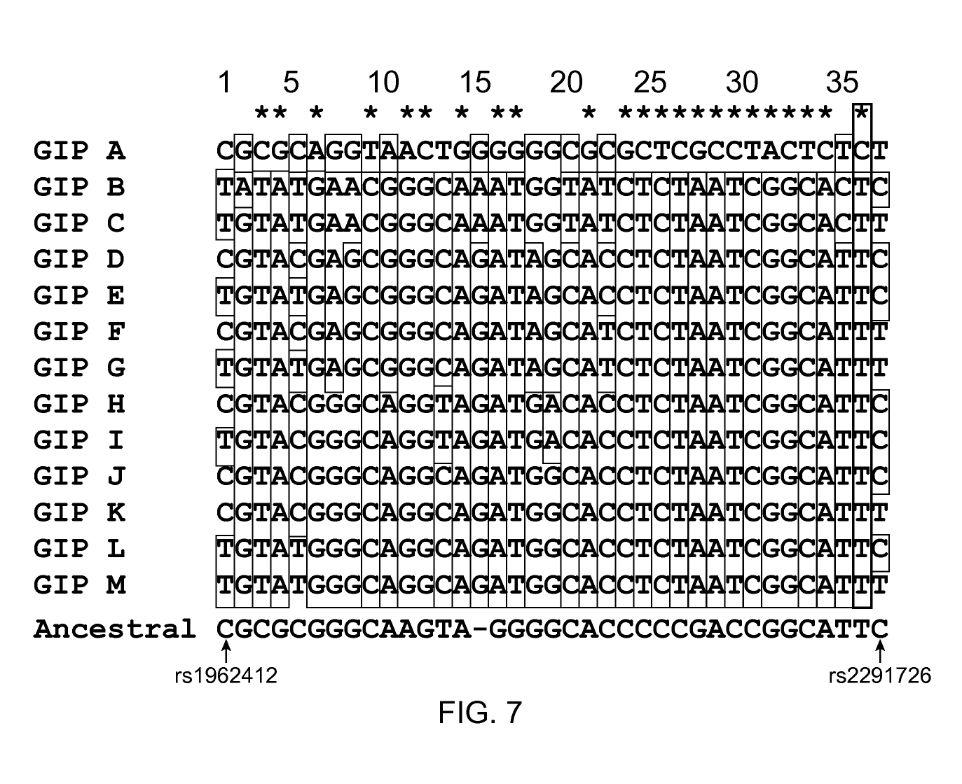

To investigate how the haplotype has evolved in different populations, we analyzed the distribution of haplotype blocks within a region of approximately 70 kb that exhibited high LD with rs2291725 in the populations of CEU and CHB+JPT (from rs196241 to rs2291726; Table 2). Among the 37 genotyped SNPs in this region, 22 are linked with rs2291725 (FIG. 7), and the overall populations are represented by 13 different haplotypes (FIG. 3, haplotypes A-M). Among these haplotypes, only haplotype A contains the derived G-44394131 allele, and only B, H, I, and K haplotypes have a frequency larger than 5% in YRI (FIG. 3). The populations in CEU and CHB+JPT are represented by 5 and 4 haplotypes, respectively. The dominant haplotype(s) is A/B in the CEU, A in the CHB+JPT, and B/H in the YRI populations (FIG. 3). An unrooted phylogenetic tree analysis based on the 13 haplotype sequences suggested that the derived haplotype A represents a diverged haplotype, whereas other haplotypes share a close relatedness. The overall data thus confirmed that the GIP locus exhibits skewed allele-frequency spectra with a long-range LD and a varied haplotype in world populations.

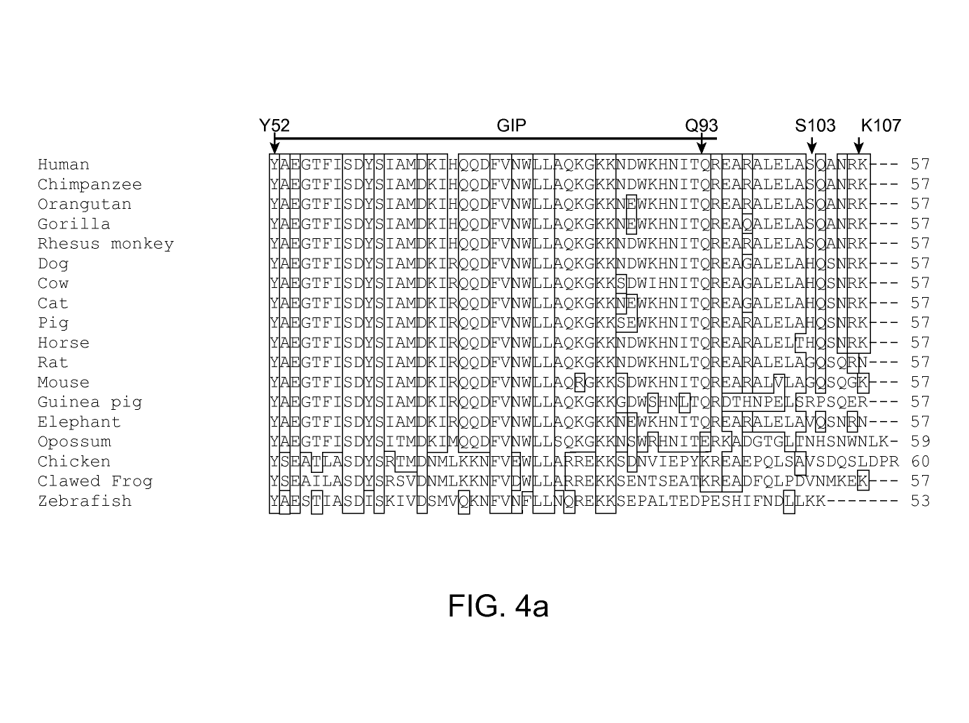

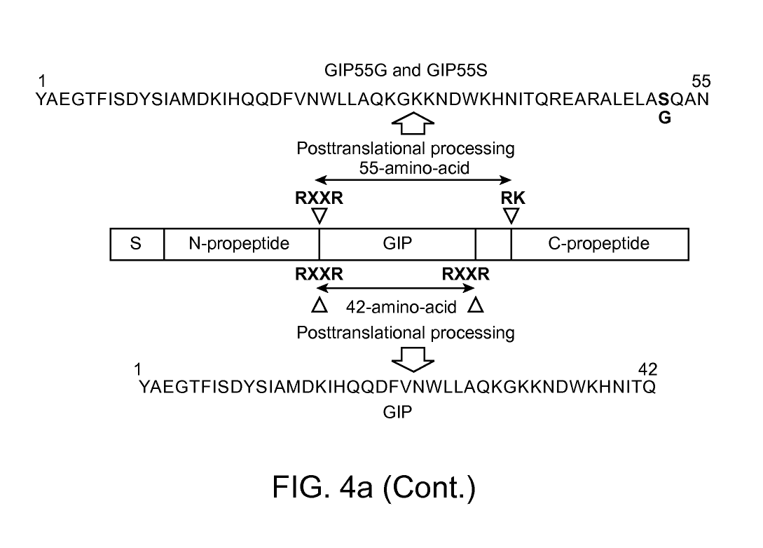

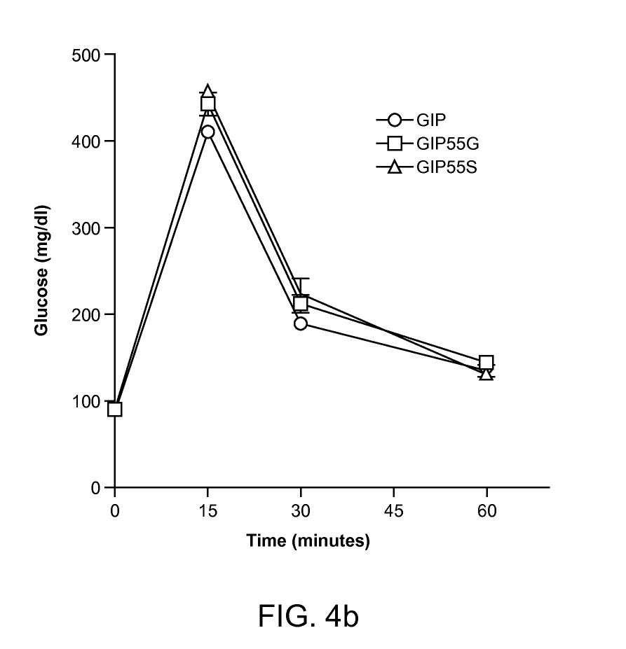

These data suggest that the high frequencies of G-44394131 in Eurasia populations can be attributed to a divergent selection of G-44394131 and A-44394131 at times postdating the separation of CEU, CHB+JPT, and YRI. The strong positive selection indicates that there might be two possible evolutionary histories. First, there could be at least four ancient haplotypes (FIG. 3, haplotypes A, B, D, and H) in our common ancestors, and haplotypes A and H were selected by environmental factors in Eurasia and Africa populations, respectively. Second, the derived haplotype A might have evolved more recently, and was introduced into African populations later. This alternative scenario is supported by the extended haplotype homozygosity found in the YRI chromosomes carrying the derived G-44394131 allele (FIG. 2d, left upper panel). Regardless of the timing of the emergence of the derived G-44394131 allele, however, our analysis indicates that selection pressure is the main force allowing the G-44394131 allele to become a dominant allele in CEU and CHB+JPT.

Whereas the selection of rs2291725 and its linked SNPs could be attributed to the selection of a single SNP or a combination of SNPs, the positioning of rs2291725 in the coding region of GIP provided a tangible target for functional analyses of these variations. To explore the possibility that the variation at rs2291725 may alter the bioactivity of GIP and provide a benefit to their carriers, we investigated the function of GIP peptides which contain the variable residue (S103 or G103). GIP was originally characterized as a 42-amino-acid peptide derived from proteolytic processing at the monobasic cleavage sites at residues 51 and 94 of the GIP open reading frame. Although the variable residue at position 103 is located outside the conventional mature GIP peptide sequence (residues 52-93; FIG. 4a, upper panel), we noticed that a conserved dibasic cleavage site is located 13 amino acids downstream of conventional cleavage site in primates, cow, horse, dog, and cat, but not rodents (FIG. 4a, upper panel; residues 106-107). Thus, the downstream dibasic site in human GIP could serve as an alternative processing site for an extended GIP isoform which is 13 amino acids longer than the conventional GIP (FIG. 4a, lower panel; GIP55G and GIP55S for G-44394131 and A-44394131, respectively). Because the receptor-activation domain of GIP is located at the N-terminus, we considered that alternative processing at the C-terminus of GIP peptide is unlikely to decimate the bioactivity of GIP. Indeed, functional testing of synthetic GIP isoforms (GIP, GIP55G, and GIP55S) in vivo showed that, similar to the conventional mature GIP, the extended GIP55G and GIP55S are biologically active, and suppress hyperglycemias to similar extents in a time-dependent manner in fasting rats (FIG. 4b).

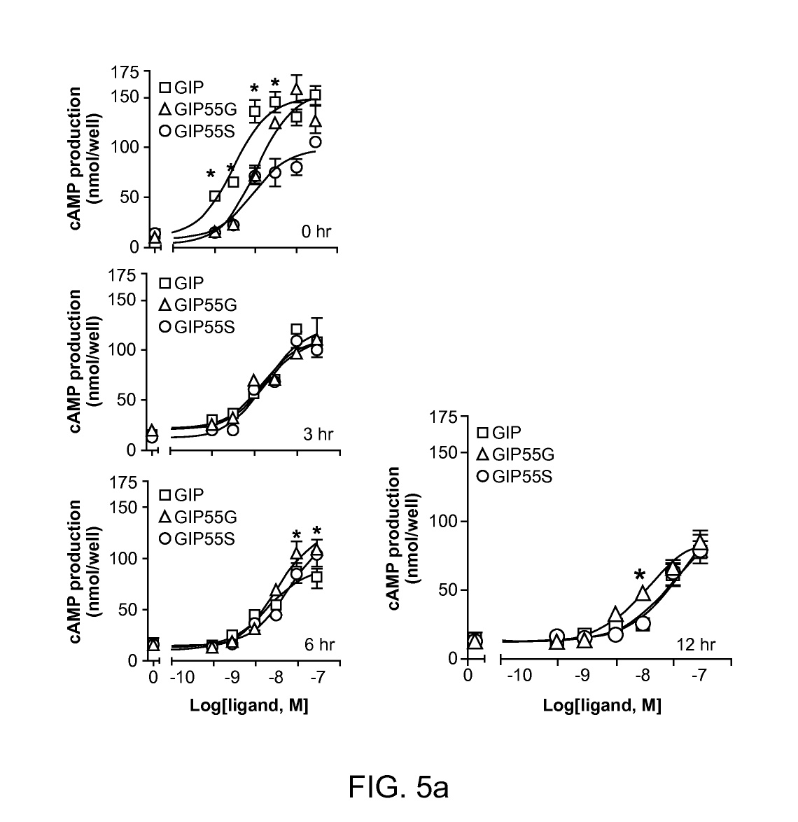

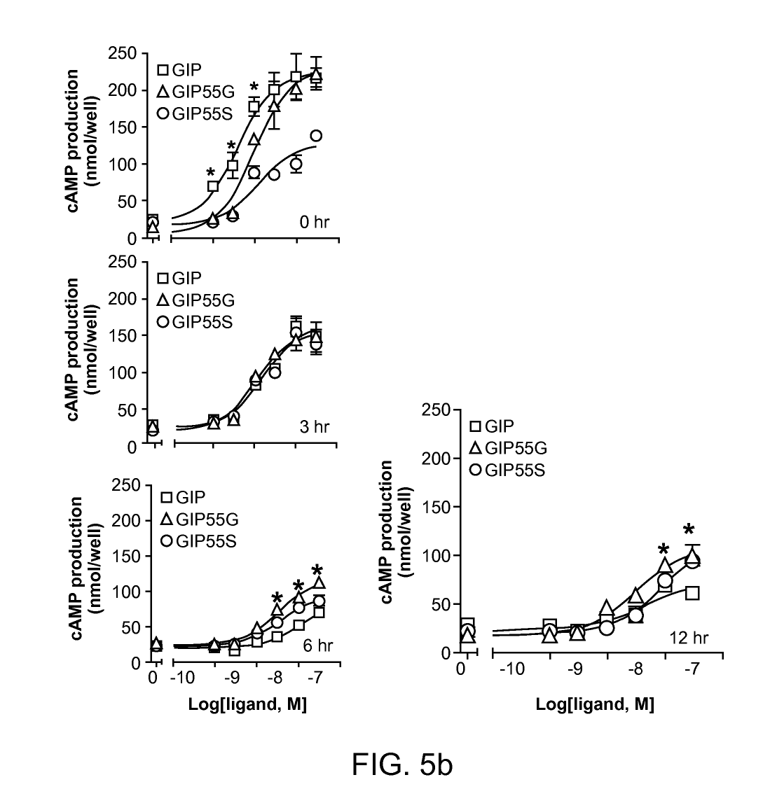

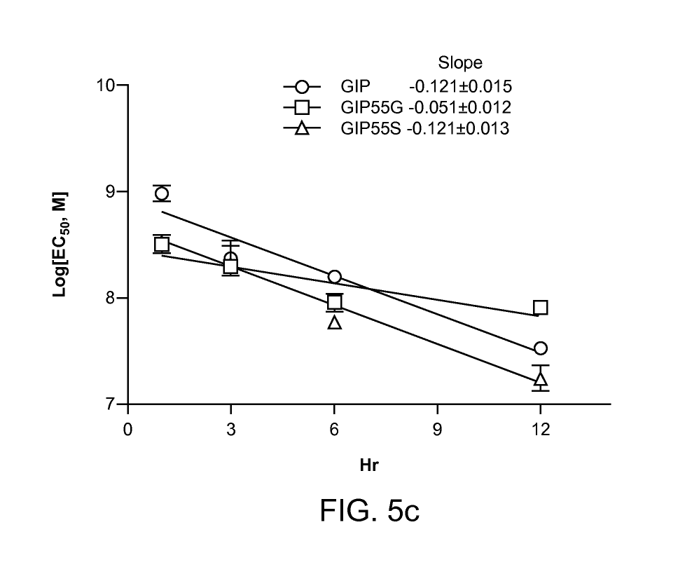

To compare the bioactivity of GIP isoforms and variants, we performed in vitro receptor-activation assays based on the stimulation of cAMP production in HEK293T cells expressing a recombinant GIP receptor (GIPR). As expected, treatments of GIPR-expressing cells with GIP lead to dose-dependent increases of cAMP production in transfected cells (FIG. 5a, top panel). Unlike the conventional GIP which exhibits an EC₅₀ of ˜0.9±0.21 nM, the extended GIP isoforms have ˜3-fold lower potencies (GIP55G, 3.2±0.21 nM; GIP55S, 2.6±0.23 nM; Table 4). Importantly, we found that GIP55G consistently increases cAMP production to significantly higher levels at higher dosages as compared to GIP55S (FIG. 5a, top panel).

Because GIP is known to be susceptible to serum degradation in vivo, we also studied the stability of GIP isoforms in human serum in vitro. Surprisingly, we found that both GIP55G and GIP55S are significantly more resistant to degradation by either pooled normal human serum (FIG. 5a) or pooled complement-preserved human serum (FIG. 5b). Whereas GIP is more potent than GIP55G and GIP55S without serum treatment (FIGS. 5a and 5b, top panel, 0 hr), the capability of GIP to stimulate cAMP production after serum incubation was significantly reduced as compared to that of GIP55G and GIP55S. The ranking of potency shifted from GIP>GIP55G>GIP55S at 0 hr to GIP=GIP55G=GIP55S at 6 hr, and GIP55G>GIP55S≧GIP at 12 hr after treatments with either pooled normal human serum (FIGS. 5a) or pooled complement preserved human serum (FIG. 5b). In addition, plots of EC₅₀ data in relation to the length of incubation with pooled human serum showed that the slope of changes in bioactivity for the GIP group is significantly steeper than that of GIP55G (FIG. 5c, P=0.0023). These data thus suggested that the rise in the frequency of G-44394131 in Eurasia populations could be associated with the quantitative increase in the overall in vivo potency of GIP55G.

Because GIP is essential for normal regulation of glucose and lipid metabolism, the finding that most populations traditionally associated with an agriculture society have a significantly higher frequency of the G-44394131 allele as compared to populations with a hunter-gatherer culture (FIG. 1) implies that the selection of derived allele at rs2291725 could represent an adaptation to the high starch food sources provided by an agriculture society in the Eurasia continent. It is generally accepted that changes in food sources present strong selection pressure during transition of human cultures. The rapid selection of genes in responses to diet changes in humans is not unprecedented. For example, the ability to digest lactose in milk (lactase persistence) has been independently selected in multiple populations that practiced a pastoral culture. Likewise, selective pressure has acted on the copy number variation of amylases. Individuals from populations with high-starch diets have more copies of amylases than those with low-starch diets, perhaps due to the improvement in the digestion of starchy foods. It has long been hypothesized that our primate ancestors ate a low-carbohydrate, high-protein diet, and that the adaptive response to low-carbohydrate intake is insulin resistance, perhaps for coping with a low dietary glucose. Conceptually, the ancestral A-44394131 allele represents a “thrifty genotype.”

The potent serum-resistant GIP55G encoded by G-44394131 may be harmful in individuals frequently facing episodes of hunger by causing hypoglycemia and lower glucose supply to the brain, thereby imposing a selection against G-44394131 in hunter-gatherer culture. Unlike the hunter-gatherer society, an agriculture society provides a stable supply of high starch staples with a different quality and a reduced need for physical mobility and metabolic efficiency. Consequently, this may lead to enhanced hyperglycemia and shifts in long-term energy balance, thereby exerting strong selection against individuals who do not have a potent serum-resistant GIP hormone to supplant the deleterious effect of hyperglycemia. This hypothesis is supported by the observation that South America populations that practiced the hunter-gatherer culture until recently have a significantly higher frequency of the ancestral A-44394131 allele as compared to their East Asia kin, Siberia Yakut (FIG. 1, Table 3; A-44394131 frequency=0.24 in Yakut vs. >0.75 in South America Indians)31. The dramatic contrast of allele frequency in these related populations thus raises the possibility that the selection at rs2291725 could be swift when the culture changed. Future studies of large samples from these populations would be necessary to power this hypothesis and reveal whether the reversal of allele frequency in South America populations is strictly due to selection, or a combination of bottleneck and environment factors.

In addition, as a target of natural selection, our study raises the possibility that rs2291725 could be a disease-risk allele. Therefore, studies of this variation in relation to susceptibility to diabetes and obesity could shed light on the relationship between ongoing changes in our diets toward refined and high energy convenience food and the emerging diabetes and obesity pandemic in modern society. Finally, it is important to note that our study reveals that humans could simultaneously contain three different GIP isoforms (A/G-44394131 (GIP+GIP55S+GIP55G), A/A-44394131 (GIP+GIP55S), and G/G-44394131 (GIP+GIP55G)) with distinct bioactivity profiles. Future study of how this mix of GIP peptides regulates glucose and lipid homeostasis in response to different diets is crucial to a better understanding of this important regulatory circuit in the development of various metabolic syndromes among human populations.

### Methods

Genotype analysis. Analysis of Fₛₜ, LD, and iHS scores. We calculated and analyzed Fₛₜ values of HapMap and HGDP-CEPH genotype data as described (http://spsmart.cesga.es)₂₁. The iHS scores, Fay and Wu's H, and extended haplotype homozygosity analyses were performed using the HapMap's phase II data set as described earlier (http://haplotter.uchicago.edu/selection)₆. LD plots for SNPs within the ALCOCO2, ATP5G1, UBE2Z, SNF8, GIP, and IGF2BP1 gene regions from rs1422645 to rs8069452, spanning 200 kb, were generated using the HaploView 3.2.

### Phenotype Tests

Reagents. Synthetic GIP peptides were obtained from the Genescript Corp. and the American Peptide Company Inc. The extended GIP55G and GIP55S isoforms were synthesized by the Stanford University Protein and Nucleic Acid Biotechnology Facility and the GL Biochem (Shanghai) Ltd. Pooled normal human serum and pooled complement preserved human serum were obtained from the Innovative Research. Stocks of different hormones were prepared in phosphate-buffered saline (PBS) and diluted in serum-free culture medium. In addition to routine chemistry and mass-spectrometry assessments, we verified the quantity of different GIP isoforms using a human GIP ELISA kit (Linco Research, Inc.).

The in vivo glucose suppression assay. Eight-week-old Sprague-Dawley rats were fasted overnight for 20 hours. To measure the ability of GIP isoforms on reducing blood glucose levels in vivo, fasting rats were injected with a select GIP peptide (100 nmoles/kg) dissolved in 0.8 ml of PBS together with glucose (3.8 g/kg body weight). Bloods were obtained via the tail vein at select time points after intraperitoneal injection of glucose with a peptide. Glucose levels in the blood were measured by an OneTouch Ultra Blood Glucose Monitoring System and OneTouch Ultra Test Strips (Johnson and Johnson).

Vector, transfection, and receptor-activation assay. Expression vector for the wild type GIP receptor was obtained from the Origene Corp. Expression constructs were transfected into the HEK293T cells using the lipofectamine 2000. To quantify resistance to serum degradation by GIP isoforms, aliquots of GIP peptides were incubated with human serum in a final concentration of 10 μM using microfuges and incubated at 37 C for indicated time-spans. The treated peptides were frozen at −80 C before testing with transfected cells. Receptor-activation activities were assayed based on cAMP production in transfected cells, and were performed as described44. The receptor-activation results were analyzed using the GraphPad Prism 5 package.

TABLE 1
Human GIP SNP rs2291725 exhibits a high population differentiation characteristic.

| | Ch. No. | Allele Frequency | | | | | Fay and Wu's | |
|---|---|---|---|---|---|---|---|---|
| Population | (2N) | A(S103) | G(G103) | Fₛₜ | | | H | iHS |
| All | 418 | 0.524 | 0.476 | | | | | |
| | | | | vs. | | | | |
| | | | | YRI | CEU | CHB + JPT | | |
| YRI | 120 | 0.950 | 0.050 | — | 0.24 | 0.48* | −11.63 | 0.953 |
| CEU | 120 | 0.517 | 0.483 | 0.24 | — | 0.08 | −10.17 | 0.556 |
| CHB + JPT | 178 | 0.245 | 0.755 | 0.48* | 0.08 | — | −45.03 | −0.828 |

Genotypes of individuals from the International HapMap Project (phase II data) were analyzed as described using the SPSmart v3 and HapPlotter
*P < 0.05.

TABLE 2
List of genotyped single nucleotide polymorphisms (SNPs) from 44246961 to 44542055 on chromosome 17.

| # | Name | Position | ObsHET | PredHET | HWpval | % Geno | FamTrio | MendErr | MAF | Alleles |
|---|---|---|---|---|---|---|---|---|---|---|
| 1 | rs674310 | 44246961 | 0.483 | 0.5 | 0.9497 | 100.0 | 0 | 0 | 0.492 | C:G |
| 2 | rs674755 | 44247046 | 0.483 | 0.5 | 0.9497 | 100.0 | 0 | 0 | 0.492 | A:T |
| 3 | rs7208074 | 44249072 | 0.467 | 0.478 | 1.0 | 100.0 | 0 | 0 | 0.383 | G:A |
| 4 | rs532154 | 44251363 | 0.467 | 0.439 | 0.752 | 100.0 | 0 | 0 | 0.483 | A:G |
| 5 | rs639679 | 44254253 | 0.267 | 0.275 | 1.0 | 100.0 | 0 | 0 | 0.167 | A:G |
| 6 | rs606911 | 44263227 | 0.267 | 0.275 | 1.0 | 100.0 | 0 | 0 | 0.167 | G:A |
| 7 | rs8076178 | 44263279 | 0.1 | 0.095 | 1.0 | 100.0 | 0 | 0 | 0.05 | G:C |
| 8 | rs12450440 | 44266352 | 0.1 | 0.095 | 1.0 | 100.0 | 0 | 0 | 0.05 | A:T |
| 9 | rs8080446 | 44267435 | 0.1 | 0.095 | 1.0 | 100.0 | 0 | 0 | 0.05 | A:G |
| 10 | rs8078984 | 44267550 | 0.1 | 0.095 | 1.0 | 100.0 | 0 | 0 | 0.05 | G:A |
| 11 | rs8078992 | 44267572 | 0.1 | 0.095 | 1.0 | 100.0 | 0 | 0 | 0.05 | G:T |
| 12 | rs7207087 | 44272952 | 0.35 | 0.45 | 0.1338 | 100.0 | 0 | 0 | 0.342 | C:G |
| 13 | rs612720 | 44278205 | 0.45 | 0.5 | 0.561 | 100.0 | 0 | 0 | 0.492 | G:A |
| 14 | rs497270 | 44273773 | 0.267 | 0.278 | 1.0 | 100.0 | 0 | 0 | 0.167 | A:G |
| 15 | rs12945070 | 44277734 | 0.367 | 0.455 | 0.1974 | 100.0 | 0 | 0 | 0.35 | G:C |
| 16 | rs533842 | 44278383 | 0.267 | 0.278 | 1.0 | 100.0 | 0 | 0 | 0.167 | G:A |
| 17 | rs12948015 | 44279979 | 0.483 | 0.493 | 1.0 | 100.0 | 0 | 0 | 0.442 | G:T |
| 18 | rs3744604 | 44280858 | 0.1 | 0.095 | 1.0 | 100.0 | 0 | 0 | 0.05 | G:A |
| 19 | rs2303015 | 44284907 | 0.1 | 0.095 | 1.0 | 100.0 | 0 | 0 | 0.05 | T:C |
| 20 | rs8074034 | 44287297 | 0.483 | 0.493 | 1.0 | 100.0 | 0 | 0 | 0.442 | A:G |
| 21 | rs1422645 | 44294657 | 0.35 | 0.45 | 0.1338 | 100.0 | 0 | 0 | 0.342 | C:G |
| 22 | rs11434 | 44296244 | 0.1 | 0.095 | 1.0 | 100.0 | 0 | 0 | 0.05 | G:A |
| 23 | rs7222365 | 44296016 | 0.12 | 0.095 | 1.0 | 100.0 | 0 | 0 | 0.05 | C:T |
| 24 | rs11654895 | 44297938 | 0.1 | 0.095 | 1.0 | 100.0 | 0 | 0 | 0.05 | A:G |
| 25 | rs12450892 | 44300502 | 0.1 | 0.095 | 1.0 | 100.0 | 0 | 0 | 0.05 | C:T |
| 26 | rs12450565 | 44300865 | 0.1 | 0.095 | 1.0 | 100.0 | 0 | 0 | 0.05 | G:C |
| 27 | rs12453671 | 44300832 | 0.1 | 0.095 | 1.0 | 100.0 | 0 | 0 | 0.05 | A:T |
| 28 | rs582150 | 44301645 | 0.483 | 0.433 | 1.0 | 100.0 | 0 | 0 | 0.442 | A:G |
| 29 | rs12449856 | 44301996 | 0.133 | 0.124 | 1.0 | 100.0 | 0 | 0 | 0.067 | C:T |
| 30 | rs5078826 | 44302434 | 0.483 | 0.493 | 1.0 | 100.0 | 0 | 0 | 0.442 | A:G |
| 31 | rs2411377 | 44303217 | 0.1 | 0.095 | 1.0 | 100.0 | 0 | 0 | 0.05 | G:A |
| 32 | [symbol] | 44305435 | 0.45 | 0.5 | 0.561 | 100.0 | 0 | 1 | 0.492 | G:C |
| 33 | rs176467 | 44305868 | 0.267 | 0.278 | 1.0 | 100.0 | 0 | 0 | 0.187 | A:G |
| 34 | [symbol] | 44305974 | 0.1 | 0.095 | 1.0 | 100.0 | 0 | 0 | 0.05 | C:T |
| 35 | [symbol] | 44307456 | 0.1 | 0.095 | 1.0 | 100.0 | 0 | 0 | 0.05 | G:A |
| 36 | rs9904761 | 44311809 | 0.367 | 0.455 | 0.1974 | 100.0 | 0 | 0 | 0.05 | C:G |
| 37 | rs4793930 | 44315789 | 0.367 | 0.455 | 0.1974 | 100.0 | 0 | 0 | 0.05 | A:G |
| 38 | [symbol] | [symbol] | 0.4 | 0.455 | 0.4682 | 100.0 | 0 | 0 | 0.35 | C:T |
| 39 | [symbol] | 44325508 | 0.4 | 0.444 | 0.5758 | 100.0 | 0 | 0 | 0.338 | T:A |
| 40 | rs318095 | [symbol] | 0.5 | [symbol] | 1.0 | 100.0 | 0 | 0 | 0.483 | T:C |
| 41 | rs962272 | [symbol] | 0.5 | 0.499 | 1.0 | 100.0 | 0 | 0 | 0.483 | A:G |
| 42 | [symbol] | [symbol] | 0.4 | [symbol] | 0.4682 | 100.0 | 0 | 0 | 0.35 | C:T |
| 43 | rs4294857 | [symbol] | 0.5 | 0.499 | 1.0 | 100.0 | 0 | 0 | 0.483 | G:A |
| 44 | [symbol] | [symbol] | [symbol] | [symbol] | 0.771 | 100.0 | 0 | 0 | [symbol] | G:A |
| 45 | [symbol] | [symbol] | 0.4 | [symbol] | [symbol] | 100.0 | 0 | 0 | [symbol] | G:A |
| 46 | rs46522 | [symbol] | 0.5 | 0.499 | 1.0 | 100.0 | 0 | 0 | 0.483 | C:T |
| 47 | [symbol] | [symbol] | [symbol] | [symbol] | 0.771 | 100.0 | 0 | 0 | [symbol] | [symbol] |
| 48 | rs318090 | 44346751 | 0.5 | 0.499 | 1.0 | 100.0 | 0 | 0 | 0.483 | G:A |
| 49 | rs12463374 | 44349845 | 0.5 | 0.499 | 1.0 | 100.0 | 0 | 0 | 0.483 | G:C |
| 50 | [symbol] | [symbol] | [symbol] | [symbol] | 0.771 | 100.0 | 0 | 0 | [symbol] | T:C |
| 51 | [symbol] | [symbol] | [symbol] | 0.499 | 1.0 | 100.0 | 0 | 0 | 0.483 | A:G |
| 52 | [symbol] | [symbol] | [symbol] | [symbol] | 0.4682 | 100.0 | 0 | 0 | 0.35 | [symbol] |
| 53 | [symbol] | 44360192 | 0.5 | 0.439 | 1.0 | 100.0 | 0 | 0 | 0.483 | A:G |
| 54 | rs1857897 | 44360508 | 0.5 | 0.499 | 1.0 | 100.0 | 0 | 0 | 0.483 | T:G |
| 55 | [symbol] | [symbol] | 0.217 | [symbol] | [symbol] | 100.0 | 0 | 0 | 0.108 | G:A |
| 56 | [symbol] | [symbol] | 0.1 | [symbol] | 1.0 | 100.0 | 0 | 0 | 0.05 | G:A |
| 57 | [symbol] | [symbol] | 0.4 | [symbol] | 0.4682 | 100.0 | 0 | 0 | 0.35 | C:T |
| 58 | rs4793992 | 44363206 | 0.5 | 0.499 | 1.0 | 100.0 | 0 | 0 | 0.483 | A:G |
| 59 | [symbol] | [symbol] | [symbol] | [symbol] | 0.4682 | 100.0 | 0 | 0 | [symbol] | C:T |
| 60 | rs17708633 | [symbol] | 0.5 | 0.499 | 1.0 | 100.0 | 0 | 0 | 0.483 | C:G |
| 61 | rs1994970 | 44363126 | 0.5 | 0.499 | 1.0 | 100.0 | 0 | 0 | 0.483 | T:C |
| 62 | rs9747645 | 44371881 | 0.5 | 0.499 | 1.0 | 100.0 | 0 | 0 | 0.483 | C:T |
| 63 | [symbol] | [symbol] | 0.5 | 0.499 | 1.0 | 100.0 | 0 | 0 | 0.483 | T:C |
| 64 | [symbol] | 44373024 | 0.5 | 0.499 | 1.0 | 100.0 | 0 | 0 | 0.483 | A:G |
| 65 | [symbol] | [symbol] | 0.5 | 0.499 | 1.0 | 100.0 | 0 | 0 | 0.483 | A:C |
| 66 | [symbol] | [symbol] | 0.5 | 0.499 | 1.0 | 100.0 | 0 | 0 | 0.483 | T:C |
| 67 | [symbol] | [symbol] | 0.5 | 0.499 | 1.0 | 100.0 | 0 | 0 | 0.483 | C:T |
| 68 | rs118779844 | [symbol] | 0.5 | 0.499 | 1.0 | 100.0 | 0 | 0 | 0.483 | G:A |
| 69 | rs12602933 | [symbol] | 0.5 | 0.499 | 1.0 | 100.0 | 0 | 0 | 0.483 | G:C |
| 70 | [symbol] | 44384903 | 0.5 | 0.499 | 1.0 | 100.0 | 0 | 0 | 0.483 | C:T |
| 71 | rs12602746 | [symbol] | 0.5 | 0.499 | 1.0 | 100.0 | 0 | 0 | 0.483 | A:C |
| 72 | [symbol] | [symbol] | [symbol] | [symbol] | 0.4682 | 100.0 | 0 | 0 | 0.35 | [symbol] |
| 73 | rs2291725 | 44394131 | 0.5 | 0.499 | 1.0 | 100.0 | 0 | 0 | 0.483 | T:C |
| 74 | [symbol] | [symbol] | [symbol] | [symbol] | 1.0 | 100.0 | 0 | 0 | [symbol] | [symbol] |
| 75 | rs8078510 | 44400861 | 0.383 | 0.439 | 0.448 | 100.0 | 0 | 0 | 0.325 | G:A |
| 76 | rs937301 | 44401275 | 0.483 | 0.5 | 0.9497 | 100.0 | 0 | 0 | 0.492 | A:G |
| 77 | rs3848460 | 44402113 | 0.483 | 0.5 | 0.9497 | 100.0 | 0 | 0 | 0.492 | A:G |
| 78 | rs3809770 | 44402595 | 0.45 | [symbol] | [symbol] | 100.0 | 0 | 0 | 0.492 | A:G |
| 79 | rs3895874 | 44402867 | 0.5 | 0.499 | 1.0 | 100.0 | 0 | 0 | 0.483 | A:G |
| 80 | [symbol] | [symbol] | 0.367 | 0.473 | 0.1246 | 100.0 | 0 | 0 | 0.383 | C:T |
| 81 | rs3894412 | 44417476 | 0.1 | 0.095 | 1.0 | 100.0 | 0 | 0 | 0.05 | T:C |
| 82 | rs4794012 | [symbol] | 0.367 | 0.464 | [symbol] | 100.0 | 0 | 0 | 0.367 | T:G |
| 83 | rs4794015 | 44422825 | 0.517 | 0.5 | 1.0 | 100.0 | 0 | 0 | [symbol] | A:G |
| 84 | rs2411755 | 44456073 | 0.533 | 0.499 | 0.8395 | 100.0 | 0 | 0 | [symbol] | T:C |
| 85 | rs1390154 | 44426482 | [symbol] | 0.469 | 0.2278 | 100.0 | 0 | 0 | 0.375 | G:A |
| 86 | rs11650936 | 44427260 | 0.55 | 0.5 | 0.6492 | 100.0 | 0 | 0 | 0.492 | G:C |
| 87 | [symbol] | 44430985 | 0.017 | 0.017 | 1.0 | 100.0 | 0 | 0 | 0.008 | C:T |
| 88 | rs1495274 | 44432647 | 0.117 | 0.139 | 0.5523 | 100.0 | 0 | 0 | 0.075 | G:A |
| 89 | rs1994969 | 44435430 | 0.4 | [symbol] | 0.2132 | 100.0 | 0 | 0 | [symbol] | T:G |
| 90 | rs9674544 | 44439710 | 0.417 | 0.493 | 0.3127 | 100.0 | 0 | 0 | 0.442 | A:G |
| 91 | rs12939375 | 44444716 | 0.4 | [symbol] | 0.2132 | 100.0 | 0 | 0 | 0.433 | G:C |
| 92 | rs6504592 | 44445297 | [symbol] | 0.124 | 1.0 | 100.0 | 0 | 0 | 0.067 | C:G |
| 93 | rs11079849 | 44445784 | [symbol] | 0.489 | 1.0 | 100.0 | 0 | 0 | 0.425 | C:T |
| 94 | rs9906710 | 44446282 | [symbol] | 0.493 | 1.0 | 100.0 | 0 | 0 | 0.442 | C:A |
| 95 | rs9906944 | 44446419 | 0.45 | 0.483 | 0.7385 | 100.0 | 0 | 0 | 0.408 | C:T |
| 96 | rs10853104 | [symbol] | [symbol] | 0.498 | 0.417 | 100.0 | 0 | 0 | 0.467 | T:C |
| 97 | rs11079851 | 44447309 | 0.4 | 0.491 | 0.2132 | 100.0 | 0 | 0 | 0.433 | C:G |
| 98 | rs4794018 | 44448397 | 0.483 | 0.493 | 1.0 | 100.0 | 0 | 0 | 0.442 | T:C |
| 99 | rs8076012 | 44456988 | 0.417 | 0.5 | 0.271 | 100.0 | 0 | 0 | 0.492 | A:G |
| 100 | rs17708997 | 44457037 | 0.15 | 0.139 | 1.0 | 100.0 | 0 | 0 | 0.075 | A:G |
| 101 | rs9912906 | [symbol] | 0.417 | 0.477 | 0.44 | 100.0 | 0 | 0 | 0.392 | A:T |
| 102 | [symbol] | 44461877 | 0.417 | 0.477 | 0.44 | 100.0 | 0 | 0 | 0.392 | T:G |
| 103 | rs4794019 | 44462289 | 0.417 | 0.477 | 0.44 | 100.0 | 0 | 0 | 0.392 | G:T |
| 104 | [symbol] | 44464426 | 0.25 | 0.219 | 0.7369 | 100.0 | 0 | 0 | 0.125 | C:G |
| 105 | rs4794026 | [symbol] | 0.417 | 0.477 | 0.44 | 100.0 | 0 | 0 | 0.392 | C:T |
| 106 | [symbol] | 44469032 | 0.433 | 0.498 | 0.417 | 100.0 | 0 | 0 | 0.467 | C:T |
| 107 | [symbol] | 44469391 | 0.417 | 0.477 | 0.44 | 100.0 | 0 | 0 | 0.392 | A:G |
| 108 | rs16945851 | 44469481 | 0.067 | 0.064 | 1.0 | 100.0 | 0 | 0 | [symbol] | C:T |
| 109 | [symbol] | [symbol] | 0.233 | 0.206 | [symbol] | 100.0 | 0 | 0 | 0.117 | C:T |
| 110 | rs1463762 | [symbol] | 0.233 | 0.206 | [symbol] | 100.0 | 0 | 0 | 0.117 | A:G |
| 111 | rs11872073 | 44474134 | 0.017 | 0.017 | 1.0 | 100.0 | 0 | 0 | 0.008 | G:A |
| 112 | [symbol] | [symbol] | 0.417 | 0.477 | 0.44 | 100.0 | 0 | 0 | 0.392 | A:G |
| 113 | [symbol] | 44477524 | 0.417 | 0.477 | 0.44 | 100.0 | 0 | 0 | 0.392 | T:C |
| 114 | rs4643373 | 44478422 | 0.417 | 0.469 | 0.5116 | 100.0 | 0 | 0 | 0.375 | T:C |
| 115 | rs9396443 | 44480981 | 0.383 | 0.5 | 0.1046 | 100.0 | 0 | 0 | 0.492 | T:C |
| 116 | rs2969 | [symbol] | 0.317 | 0.349 | 0.6576 | 100.0 | 0 | 0 | 0.225 | C:T |
| 117 | rs11655950 | 44464130 | 0.417 | 0.477 | 0.44 | 100.0 | 0 | 0 | 0.392 | G:A |
| 118 | rs2241932 | 44486900 | 0.067 | 0.064 | 1.0 | 100.0 | 0 | 0 | [symbol] | G:C |
| 119 | [symbol] | 44486900 | 0.4 | 0.499 | 0.1751 | 100.0 | 0 | 0 | 0.483 | G:A |
| 120 | rs6504593 | 44487818 | 0.4 | 0.499 | 0.1751 | 100.0 | 0 | 0 | 0.483 | C:T |
| 121 | [symbol] | [symbol] | 0.433 | 0.5 | 0.5994 | 100.0 | 0 | 0 | 0.5 | [symbol] |
| 122 | [symbol] | 44499531 | 0.433 | 0.48 | 0.5797 | 100.0 | 0 | 0 | 0.4 | T:C |
| 123 | rs12939237 | 44499563 | 0.183 | 0.167 | 1.0 | 100.0 | 0 | 0 | 0.092 | G:A |
| 124 | rs940088 | 44500847 | [symbol] | 0.48 | 0.5797 | 100.0 | 0 | 0 | 0.4 | T:C |
| 125 | rs11656072 | 44502360 | 0.167 | 0.18 | 0.9127 | 100.0 | 0 | 0 | 0.1 | T:C |
| 126 | rs11656250 | 44512427 | 0.3 | 0.278 | 0.9897 | 100.0 | 0 | 0 | 0.167 | G:A |
| 127 | rs11657661 | 44520235 | 0.283 | 0.289 | 1.0 | 100.0 | 0 | 0 | 0.175 | C:T |
| 128 | [symbol] | 44520997 | 0.517 | 0.489 | 0.9112 | 100.0 | 0 | 0 | 0.425 | C:G |
| 129 | [symbol] | 44532746 | 0.483 | 0.477 | 1.0 | 100.0 | 0 | 0 | 0.392 | C:T |
| 130 | rs9303547 | 44532912 | 0.267 | 0.231 | 0.6317 | 100.0 | 0 | 0 | 0.133 | A:T |
| 131 | rs9910632 | 44537708 | 0.433 | 0.464 | 0.7541 | 100.0 | 0 | 0 | 0.367 | C:T |
| 132 | rs1523135 | [symbol] | 0.433 | 0.464 | 0.7541 | 100.0 | 0 | 0 | 0.367 | G:C |
| 133 | rs6504595 | 44542055 | 0.3 | 0.339 | 0.5365 | 100.0 | 0 | 0 | 0.217 | G:T |

[symbol]

indicates data missing or illegible when filed

TABLE 3
Allele frequency of rs2291725 in
the HGDP-CEPH populations.

| | Allele Frequency (%) | | |
|---|---|---|---|
| | A- | G- | Chromo- |
| | 443944131 | 44394131 | some |
| AFRICA (Sub-Sahara) | | | |
| C. African Republic- | 100.0 | 0.0 | 44 |
| Biaka-Pygmy | | | |
| D.R. of Congo- | 92.2 | 3.6 | 26 |
| Mbuti Pygmy | | | |
| Kenya-Bantu | 100.0 | 0.0 | 22 |
| Namidia-San | 100.0 | 0.0 | 10 |
| Nigeria-Yoruba | 90.5 | 9.5 | 42 |
| Senegal-Mandenka | 93.2 | 6.8 | 44 |
| South Africa-Bantu | 93.7 | 6.3 | 16 |
| AMERICA | | | |
| Brazil-Karitiana | 75.0 | 25.0 | 28 |
| Brazil-Surui | 75.0 | 25.0 | 16 |
| Colombia-Piapoco | 78.6 | 21.4 | 14 |
| and Curripaco | | | |
| Mexico-Maya | 71.4 | 28.6 | 42 |
| Mexico-Pima | 57.1 | 42.9 | 28 |
| EUROPE | | | |
| France-Basque | 58.3 | 41.7 | 48 |
| France-French | 48.2 | 51.8 | 56 |
| Italy-Sardinian | 39.3 | 60.7 | 56 |
| Italy-Tuscan | 50.0 | 50.0 | 16 |
| Italy-Bergamo | 53.8 | 46.2 | 26 |
| Orkney Islands-Orcadian | 43.3 | 56.7 | 30 |
| Russia Caucasus-Adygei | 33.0 | 67.0 | 34 |
| Russia-Russian | 52.0 | 48.0 | 50 |
| MIDDLE EAST | | | |
| Algeria (Mzab)-Mozabite | 79.3 | 20.7 | 58 |
| Israel (Carmel)-Druze | 45.2 | 54.8 | 84 |
| Israel (central)-Palestinian | 56.5 | 43.5 | 92 |
| Israel (Negev)-Bedouin | 45.7 | 54.3 | 92 |
| CENTRAL-SOUTH ASIA | | | |
| China-Uygur | 55.0 | 45.0 | 20 |
| Pakistan-Balochi | 47.9 | 52.1 | 48 |
| Pakistan-Brahui | 44.0 | 56.0 | 50 |
| Pakistan-Burusho | 52.0 | 48.0 | 50 |
| Pakistan-Hazara | 38.6 | 61.4 | 44 |
| Pakistan-Kalash | 56.5 | 43.5 | 46 |
| Pakistan-Makrani | 48.0 | 52.0 | 50 |
| Pakistan-Pathan | 45.5 | 54.5 | 44 |
| Pakistan-Sindhi | 64.6 | 35.4 | 48 |
| OCEANIA | | | |
| Bougainville- | 27.3 | 72.7 | 22 |
| NAN Melanesian | | | |
| New Guinea-Papuan | 58.8 | 41.2 | 34 |
| EAST ASIA | | | |
| Cambodia-Cambodian | 40.0 | 60.0 | 20 |
| China-Dai | 45.0 | 55.0 | 20 |
| China-Daur | 38.9 | 61.1 | 18 |
| China-Han | 28.4 | 71.6 | 88 |
| China-Hezhen | 27.8 | 72.2 | 18 |
| China-Lahu | 31.2 | 68.8 | 16 |
| China-Miaozu | 40.0 | 60.0 | 20 |
| China-Mangola | 30.0 | 70.0 | 20 |
| China-Naxi | 62.5 | 37.5 | 16 |
| China-Oroquen | 22.2 | 77.8 | 18 |
| China-She | 30.0 | 70.0 | 20 |
| China-Tu | 35.0 | 65.0 | 20 |
| China-Tujia | 35.0 | 65.0 | 20 |
| China-Xibo | 5.6 | 94.4 | 18 |
| China-Yizu | 35.0 | 65.0 | 20 |
| Japan-Japanese | 21.4 | 78.6 | 56 |
| Siberia-Yakut | 24.0 | 76.0 | 50 |
| Total | | | 1888 |

TABLE 4
EC50 values (-Log EC50, mean ± SEM) for GIP, GIP55G, and GIP55S.

| | | | |
|---|---|---|---|
| Hours of incubation | GIP | GIP55G | GIP55S |

With pooled human serum

| | | | |
|---|---|---|---|
| 0 | 9.02 ± 0.15 | 8.47 ± 0.14 | 8.56 ± 0.19 |
| 3 | 8.21 ± 0.13 | 8.32 ± 0.19 | 8.34 ± 0.14 |
| 6 | 8.21 ± 0.15 | 8.02 ± 0.10 | 7.74 ± 0.14 |
| 12 | 7.52 ± 0.19 | 7.93 ± 0.09 | 7.38 ± 0.16 |

With pooled complement preserved human serum

| | | | |
|---|---|---|---|
| 0 | 8.91 ± 0.15 | 8.51 ± 0.14 | 8.43 ± 0.18 |
| 3 | 8.53 ± 0.16 | 8.22 ± 0.15 | 8.49 ± 0.13 |
| 6 | 8.18 ± 0.12 | 7.88 ± 0.09 | 7.79 ± 0.13 |
| 12 | 7.53 ± 0.17 | 7.86 ± 0.14 | 7.12 ± 0.22 |

### EXAMPLE 2

### Regulatory GIP Variants Underlie Susceptibility to Abnormal Glucose Metabolism During Pregnancy

To explore genes that underlie the varied diabetes-associated traits in humans, we searched for genetic variants that exhibit a high population differentiation and are located in gene regions previously implicated in the regulation of carbohydrate and lipid metabolism. Here, we identified three linked SNPs at the 5′ gene region of the glucose-dependent insulinotropic polypeptide (GIP; rs3895874, rs3848460, and rs937301) as targets of selection in East Asian. Interestingly, in vitro promoter assays showed that reporters carrying an ancestral GIP haplotype (GIP^{-1920G}) exhibit significantly higher transcriptional activities than that with a derived GIP haplotype (GIP^{-1920A}) in transfected cells, representing a selective mechanism for the population genetic observations. Consistently, measurements of serum glucose levels in pregnant women undertaking a screening glucose challenge test showed that patients with a homozygous GIP^{-1920A/A} genotype have significantly higher serum glucose levels at 1 hr post-test and a significantly higher incidence of glucose intolerance when compared to patients with a homozygous GIP^{-1920G/G} genotype. When controlled for variations in GIPR, a significant difference in glucose levels was also observed between the GIP^{-1920G/G} and GIP^{-1920G/A} genotype groups. Taken together, our study indicated that common regulatory variants in the GIP promoter represent causal mutations underlying the selection of long GIP haplotypes, and impart a difference in susceptibility to abnormal glucose metabolism during pregnancy in select populations.

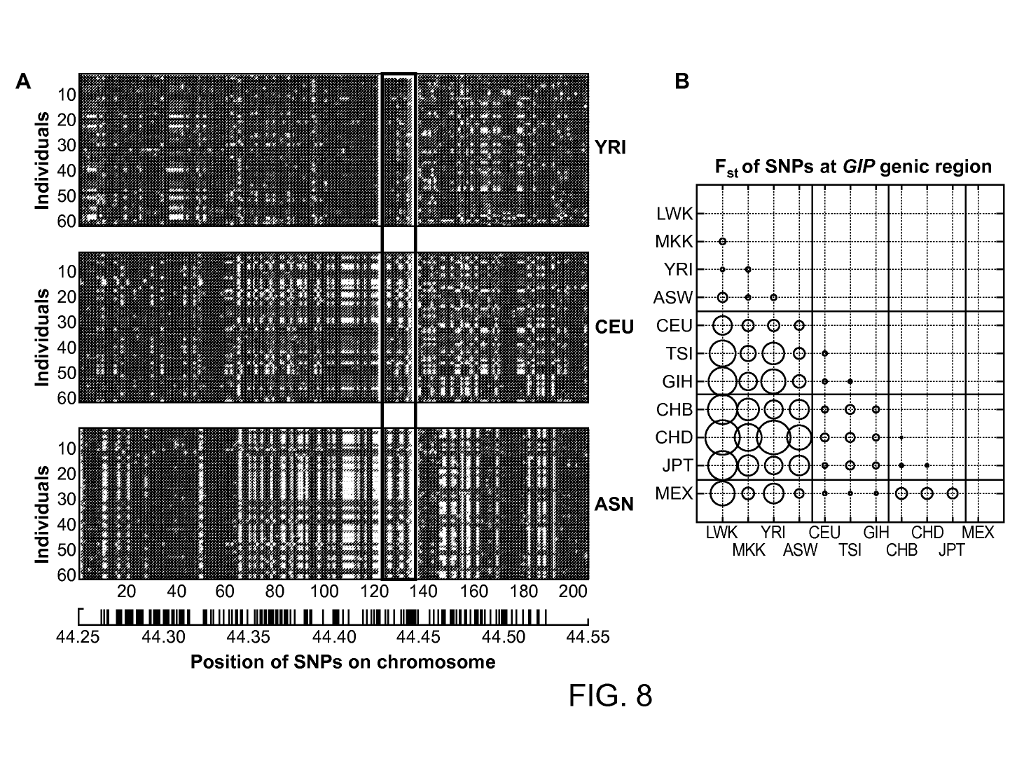

Common SNPs at the glucose-dependent insulinotropic polypeptide (GIP) locus are targets of selection in humans. Based on the screening of F^{ST} values of SNPs in 180 gene loci previously implicated in the regulation of diabetes- and obesity-related traits (Supplementary Table 5), we found that a cluster of SNPs at the GIP locus (44 SNPs from rs9904761 to rs3895874; Genome Build 36.3, human chr17:44,311 kb-44,402 kb) exhibit F^{ST} values at the top 2-8% bracket in comparisons between YRI (Yoruba from Ibadan) and ASN (pooled samples of Chinese from Beijing (CHB) and Japanese from Tokyo (JPT)) populations. In contrast, no significant population differentiation of these variants was observed in comparisons between YRI and CEU (U.S. residents with northern and western European ancestry), or between CEU and ASN. A plot of genotypes of a 250 kb region around GIP locus in the three HapMap I populations showed that SNPs surrounding GIP are highly linked in ASN and CEU, suggesting that select haplotypes became dominant in these populations (FIG. 8A, middle and lower panels). In contrast, genotypes surrounding GIP in YRI exhibited a high diversity (FIG. 8A, upper panel). To further study the difference in GIP locus among populations, we analyzed populations from HapMap III, which genotyped 1184 individuals from 11 populations. Consistently, analyses of genic SNPs at the GIP locus between 11 HapMap III populations showed that F^{ST} values are the highest between East Asian and African populations (FIG. 8B; F^{ST}=0.25-0.52 for comparisons of Han-Chinese and Japanese populations vs. Luhya in Webuye, Kenya, Maasai in Kinyawa, Kenya, or Yoruba in Ibadan, Nigeria;). The allele differentiation between other populations was minimal.

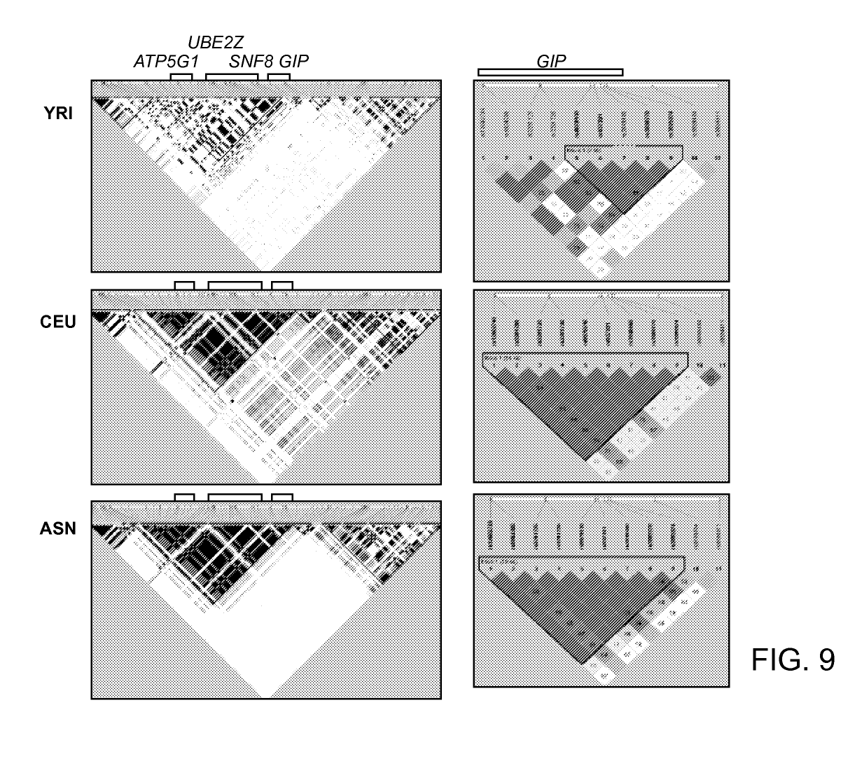

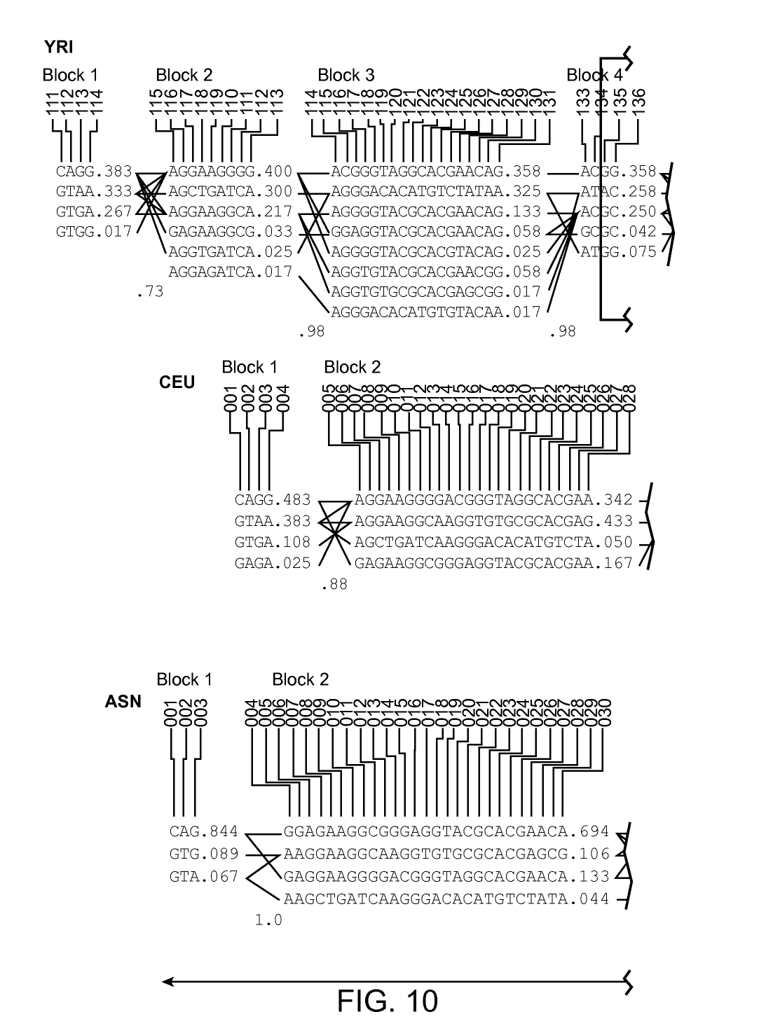

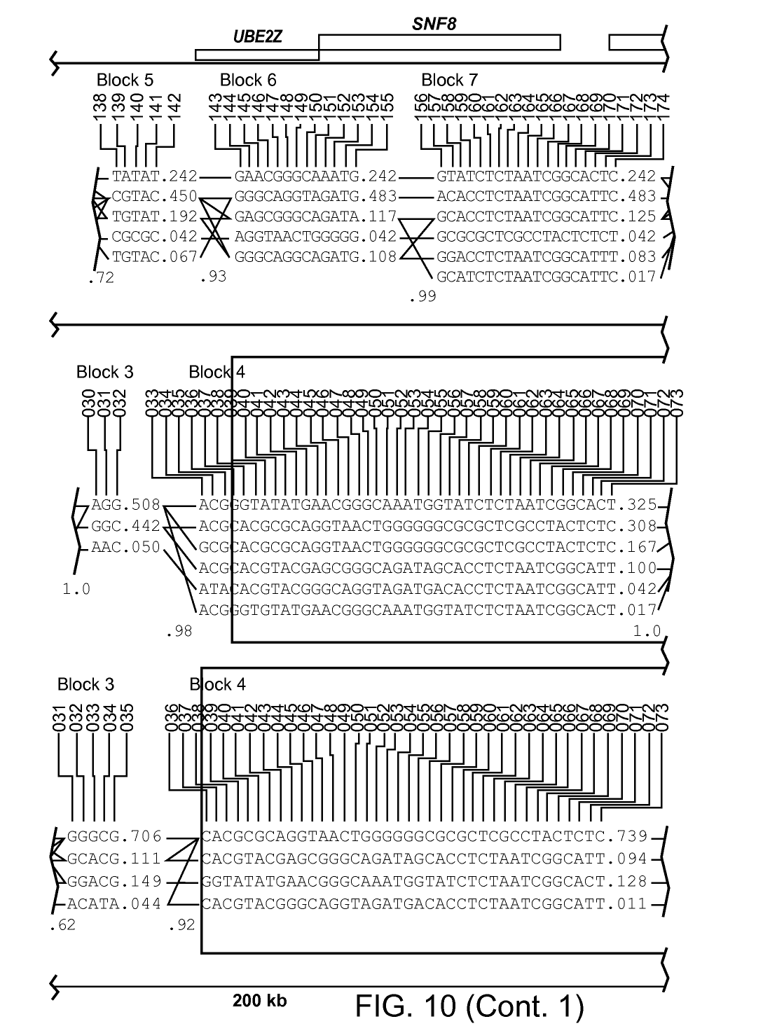

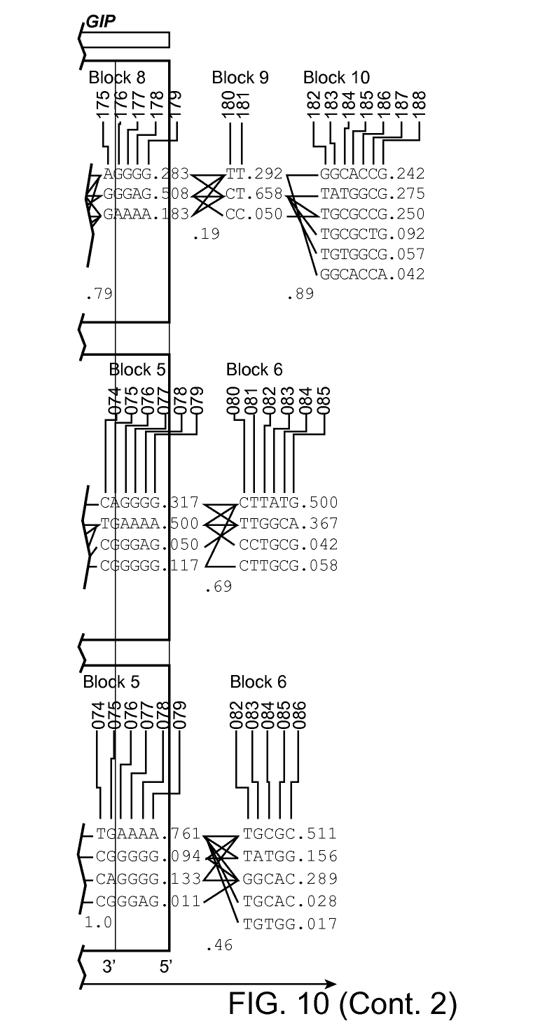

To clarify the potential development mechanism of variants at the GIP locus, we analyzed linkage disequilibrium (LD) blocks and haplotypes in the three HapMap I populations. LD analysis showed that the genomic region encompassing GIP and neighboring UBE2Z, SNF8, and ATP5G1 forms extended LD blocks in CEU and ASN chromosomes; whereas, YRI chromosomes exhibit no such LD block (FIG. 9, left panel). In both CEU and ASN, a highly linked 91 kb region surrounding GIP was represented by 6 and 4 inferred haplotypes, respectively (FIG. 10, middle and lower panels; chr17:44,311 kb-44,402 kb). In contrast, a total of 51 haplotypes were inferred in the same region in YRI chromosomes (FIG. 10, upper panel). In addition, plots depicting extended haplotype homozygosity (EHH) showed that most ASN chromosomes with the derived allele at rs3895874 (at −1920 position of GIP; referred to as GIP−^{1920G/A} mutation) have EHH extending over 200 kb. In contrast, YRI chromosomes showed minimal EHH. Furthermore, analysis of the haplotype block diversity indicated that the recombination frequency surrounding the GIP locus in ASN was significantly lower than that of YRI (FIG. 10, X² test, P<0.05), suggesting that frequencies of a few extended haplotypes increased rapidly in ASN, and perhaps CEU, after these populations split from YRI.

Also, LD and haplotype analyses indicated that four SNPs at the 5′ gene region of GIP are highly linked in an uninterrupted haploblock (rs3895874, rs3809770, rs3848460, and rs937301, at position −1920, −1650, −1158, and −320 of GIP, respectively; FIG. 9, right panel; FIG. 10). Four SNPs in this haploblock comprised three inferred haplotypes in all three populations, and these tetranucleotide polymorphisms were referred to as derived GIP−^{1920AAAA}, ancestral GIP^{-1920GAGG}, and ancestral GIP^{-1920GGGG} haplotypes. Importantly, the frequency of these haplotypes exhibited significant differences among populations (Table 5, X² test, P<0.0001). Frequency of the derived GI^{P-1920AAAA} haplotype is 18.3% in YRI, and this haplotype has a frequency of 50% and 76.1%, in CEU and ASN, respectively. By contrast, the dominant ancestral haplotype in YRI chromosomes (GIP^{-1920GAGG}, 50.8%) was only found in 1.1% of ASN chromosomes (Table 5). Taken together, these data indicated that derived GIP haplotypes exhibit characteristics of a partially dominant allele which increases in frequency rapidly, and has little time to recombine with variants in the surrounding region.

Despite these observations, two neutrality tests: iHS test and XP-EHH test, and the hitchhiking mapping approach failed to detect a statistically significant deviation from the genome-wide average. These results may underscore the limited power of these tests, designed to identify alleles that have reached near-fixation in one population, in detecting a partial sweep. Given the presence of highly differentiated alleles, high frequency derived alleles, and long derived haplotypes in select populations, we concluded that GIP locus was partially selected in ASN. To exclude the possibility that the selection of GIP haplotypes was an event of randomness (i.e., a genetic drift occurred in a population), we applied functional tests to uncover putative causal mutations at the GIP locus.

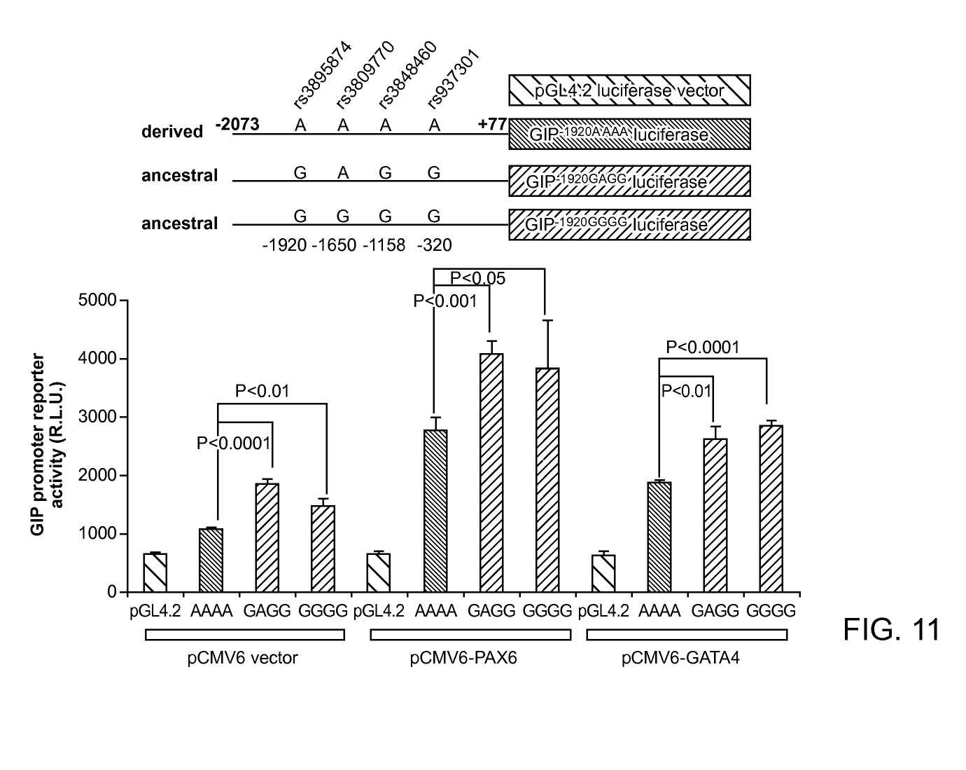

The GIP promoter reporter activity is haplotype-dependent. Whereas the selection may be attributed to any of the linked SNPs surrounding GIP, we noted that the haploblock comprising rs3895874, rs3809770, rs3848460, and rs937301 at the GIP promoter region is flanked by the LD border at the 5′ end and a hot spot for recombination at the 3′ end in all HapMap I populations, suggesting these variants could play a role in the shift of haplotype frequencies in Eurasian populations. In addition, we reasoned that variants at the GIP promoter region represent causal mutations because GIP secretion in the gastrointestinal system adjusts constantly according to the frequency and the magnitude of energy inputs whereas, positive selection increasing population differentiation mainly affects SNPs that are in the 5′ untranslated region or are nonsynonymous. To test for an allelic effect of GIP variants at the 5′ gene region, we constructed and tested GIP promoter reporters with each of the three major haplotypes in transfected HEK293T cells (FIG. 11, upper panel).

Measurement of promoter reporter activities in transfected cells showed that constructs with an ancestral haplotype (GIP^{-1920GAGG} or GIP^{-1920GGGG}) exhibit luciferase reporter activities 25-45% higher than that with a derived haplotype (GIP^{-1920AAAA}) (FIG. 11, lower panel; P<0.01). Because the GIP promoter region contains elements important for the regulation by transcriptional factors including, PAX6 and GATA4, we also determined whether the GIP promoter reporter activity is haplotype-dependent in the presence of these transcription factors. As expected, co-expression of PAX6 or GATA4 increased the basal reporter activities by 1.5-2.5 and 0.7-0.8 folds, respectively (FIG. 11, lower panel). Importantly, we found that reporters with an ancestral haplotype consistently exhibit a significantly higher activity than that containing the derived haplotype in the presence of PAX6 or GATA4 (P<0.01).

The GIP genotype is associated with serum glucose levels after an oral glucose challenge test in pregnant Han-Chinese women. The combined results of genomic and in vitro analyses suggested that variations at rs3895874, rs3848460, and rs937301, but not rs3809770, could contribute to varied GIP promoter activities among individuals. These results also raised the possibility that GIP haplotypes could affect individual's response to energy inputs under specific conditions. To be selected in the last forty thousand years after the split of YRI and CEU/ASN populations, these SNPs are expected to have effects on energy metabolism and survival at an early age. Therefore, we hypothesized that studies of glucose metabolism during pregnancy provides a window to investigate the role of GIP haplotypes in human adaptation because pregnancy represents a critical life stage that subjects individuals to excessive metabolic load at an early age, and many women suffer the adverse consequences of gestational diabetes mellitus. In addition, we hypothesized that individuals with a derived haplotype likely have an enhanced susceptibility to glucose intolerance because GIP promoter with a derived haplotype has a less robust bioactivity as compared to that with an ancestral haplotype in vitro. To test these hypotheses, we then performed genotyping analysis of Han-Chinese women undertaking a screening glucose challenge test for gestational diabetes. Because alleles at rs3809770 has a negligible effect on promoter reporter activities in vitro, patients at the 23rd to the 29th week of pregnancy were assigned to three genotype groups (GIP ^{-1920G/G}, GIP^{-1920G/A}, or GIP^{-1920A/A}) based on alleles at rs3895874, rs3848460, and rs937301 (Table 5).

Genotyping of SNPs in a panel of 123 unrelated patients showed that the frequency of GIP genotypes in these individuals is similar to that of ASN and is in Hardy-Weinberg equilibrium; frequencies of GIP^{-1920G/G}, GIP^{-1920G/A}, and GIP^{-1920A/A} genotypes were 0.106, 0.488, and 0.407, respectively (Table 5). Also, as expected, alleles at rs3895874 were in absolute LD with those of rs3848460 and rs937301. Serum glucose levels in these patients ranged from 74-230 mg/dL at 1 hr after sugar intake, and the average glucose level was 125.3±2.9 mg/dL. Importantly, we found that serum glucose levels in patients with a homozygous genotype for the derived haplotype (GIP^{-1920A/A}) is significantly higher than that of patients with a homozygous genotype for the ancestral haplotype (GIP^{-1920G/G}) (Table 5, P=0.046). A marginal significance was also observed when homozygotes GIP^{-1920A/A} were compared with heterozygotes GIP^{-1920G/A} and homozygotes GIP^{-1920G/G} combined (P=0.057; 130.9±4.3 mg/dL vs. 120.9±3.8 mg/dL). In addition, we showed that whereas 38.0% of women with a G/P^{-1920A/A} genotype have glucose levels greater than the 140 mg/dL threshold at 1 hr post-test, only 7.7% of patients with a GIP^{-1920G/G} genotype have a glucose level above the cutoff (Table 5, P=0.037). These results are concordant with in vitro assays showing that G/P^{-1920G} allele is associated with an enhanced promoter activity, and hence the capability to maintain glucose homeostasis.

We also genotyped two linked GIPR variants (rs10423928 and rs1800437; referred to as GIPR^{1159G/G} mutation in Table 5), which were shown to be associated with glucose levels after an oral glucose challenge in healthy individuals and patients in genome-wide associated studies. Unlike GIP variants, we found no association between GIPR SNPs and serum glucose levels in pregnant women (Table 5). Given the observed role of GIPR variants in genome wide association studies, we sought to isolate the potential confounding effect by restricting the serum glucose analysis to women with the dominant GIPR^{1159G/G} genotype. Interestingly, among this pool of patients we found that serum glucose levels of GIP^{-1920A/A} homozygotes are 13.5% and 26.7% higher than that of heterozygotes (P=0.049) and GIP^{-1920G/G} homozygotes (P=0.009), respectively (Table 5). These data thus confirmed that GIP^{-1920A} allele is associated with a diminished capability to maintain glucose homeostasis.

TABLE 5
Human GIP haplotype frequency and association with glucose challenge responses among
123 pregnant Han-Chinese women. For glucose challenge response analysis, GIP
genotypes were considered in three groups: GIP^{-1920G/G} (homozygotes with an ancestral
haplotype), GIP^{-1920G/A} (heterozygotes), and GIP^{-1920A/A}
(homozygotes with a derived haplotype).
Frequency of inferred GIP haplotype and genotypes in HapMap I dataset+

| Inferred haplotype | ASN (N = 174) | CEU (N = 120) | YRI (N = 114) |
|---|---|---|---|
| GIP [symbol] | 0.011 | 0.050 | 0.508 |
| GIP [symbol] | 0.228 | 0.434 | 0.283 |
| GIP [symbol] | 0.761 | 0.500 | 0.183 |

Genotype at [symbol] 895874

| | | | |
|---|---|---|---|
| GIP [symbol] | 0.081 | 0.233 | 0.386 |
| GIP [symbol] | 0.333 | 0.500 | 0.614 |
| GIP [symbol] | 0.586 | 0.267 | 0.000 |

Characteristics of the patient population

| | | | | | |
|---|---|---|---|---|---|
| | No. of patients | Average glucose | % of patients with a | Age | |
| Genotype | (frequency) | level (mg/dL) | level [symbol] 140 mg/dL | (yr) | BMI |

All patients

| | | | | | |
|---|---|---|---|---|---|
| GIP [symbol] | 13 (0.106) | 115.1 ± 6.2 | 7.7% | 32.5 ± 0.7 | 25.6 ± 0.8 |
| GIP [symbol] | 60 (0.488) | 122.9 ± 4.4 | 26.7% | 31.4 ± 0.6 | 25.3 ± 0.5 |
| GIP [symbol] | 50 (0.407) | 130.9 ± 4.3* | 38.0%* | 30.1 ± 0.7 | 26.3 ± 0.7 |
| GIPR [symbol] | 7 (0.057) | 141.7 ± 16.1 | 42.9% | 33.0 ± 2.0 | 25.9 ± 1.2 |
| GIPR [symbol] | 41 (0.333) | 122.2 ± 4.4 | 17.5% | 32.3 ± 0.7 | 26.1 ± 0.6 |
| GIPR [symbol] | 75 (0.610) | 125.5 ± 3.8 | 34.7%* | 30.2 ± 0.5 | 25.5 ± 0.5 |
| All | 123 | 125.3 ± 2.9 | 29.5% | 31.0 ± 0.4 | 25.7 ± 0.4 |

Patients with a GIPR [symbol] genotype

| | | | | | |
|---|---|---|---|---|---|
| GIP [symbol] | 8 (0.107) | 107.8 ± 7.6 | 0.0% | 32.1 ± 1.1 | 25.2 ± 1.1 |
| GIP [symbol] | 37 (0.493) | 120.3 ± 5.1 | 29.7% | 31.3 ± 0.6 | 25.0 ± 0.7 |
| GIP [symbol] | 30 (0.400) | 136.6 ± 6.3** | 50.0%* | 28.2 ± 0.9 | 26.3 ± 0.9 |
| Total | 75 | 125.5 ± 3.8 | 34.7% | 30.2 ± 0.5 | 25.6 ± 0.5 |

+Frequencies of GIP haplotypes were significantly different among all HapMap I populations (P < 0.0001).
*Significantly different from patients with the GIP^{-1920G/G} genotype (chi-square test, P < 0.05).
**Significantly different from patients with a GIP^{-1920G/G} or GIP^{-1920G/A} genotype (P < 0.05). [symbol]
indicates data missing or illegible when filed

Based on studies of pregnant women undertaking a gestational diabetes screening test, we showed that patients with a homozygous GIP^{-1920A/A} genotype have significantly higher serum glucose levels at 1 hr post-test and a higher incidence of glucose intolerance as compared to patients with a GIP^{-1920G/G} genotype. Because GIP promoter reporters carrying an ancestral haplotype exhibited a significantly higher bioactivity as compared to that with a derived haplotype, the diverged glucose challenge response among patients can be attributed to variations at the 5′ untranslated region of GIP. In addition, our data showed that these regulatory variants represent causal mutations underlying the selection of GIP haplotypes in human populations. Collectively, our study indicated that variants at the GIP promoter represent metabolism modifiers that were susceptible to the influence of environmental pressure, and emphasized the importance of understanding energy balance adaptation in the face of emerging diabetes and obesity epidemics.

While a role of common GIP variants in glucose metabolism has not been reported, several linked GIPR variants were shown to be associated with diabetes-related traits in genome wide association studies evaluating >29,000 individuals. It was speculated the nonsynonymous rs1800437 polymorphism could represent a causal mutation that influences the signal strength of GIPR. Therefore, our studies are compatible with these earlier findings that genetic variations in components of the GIP signaling pathway are relevant in shaping variations in human glucose metabolism.

Diabetes and obesity comprise heterogeneous phenotypes among ethnic groups. For example, the Asian type II diabetes patients were characterized by a lower body-mass index (BMI) and lower serum insulin levels as compared to several other ethnic groups. Likewise, the prevalence of gestational diabetes mellitus was highly population-dependent. It is possible that some of these variations owed their origins to selection in recent history. Indeed, recent analyses of human genomes showed that major human populations experienced varying degree of selection, and geographic barriers that imposed impediments to human interactions have led to a geography-based genetic substructure. In addition, it was shown that genetic variants in at least 20 gene loci are associated with glucose levels after an oral glucose challenge test and/or type II diabetes, and some of these variants exhibit high population differentiation. While the selective advantage conferred by different GIP haplotypes remains to be investigated, our finding is compatible with studies showing that Asian women have a higher incidence of gestational diabetes mellitus as compared to Caucasian women. Because impaired GIP signaling has been associated with type II diabetes and gestational diabetes mellitus, GIP haplotypes could represent novel markers to stratify the risk associated with these syndromes, thereby improving clinical outcomes. This could be particularly applicable to gestational diabetes as timely treatments of this syndrome effectively improve pregnancy outcomes.

In the present study, we chose to study East Asian patients as a proof of concept testing based on the understanding that selection pressures are most likely ongoing in populations that exhibit the most significant evidence. Given the evolutionary signatures at the GIP locus, the plausible biological mechanism, and the significant result in the Han-Chinese women, GIP variant-mediated diversification in glucose metabolism can also exist in European populations which carry a high frequency of derived alleles as well. Indeed, preliminary studies have provided evidence for an association between GIP variants and glucose metabolism in GWA studies. Therefore, our study demonstrates the role of GIP genotypes in the predisposition to different metabolic syndromes in multiple ethnic groups.

The emergence of diabetes and obesity epidemics in modern society is broadly considered to be associated with a decrease of physical activity requirement, and the availability of abundant food resource. This concept is concordant with the idea that food source represents one of the most important selection pressures during transitions of human culture, and the gradual transition from a hunter-gatherer society to an agriculture society posed a wide spectrum of pressure on the physiology of human ancestors. Given that derived GIP haplotypes represent stationary haplotypes in all HapMap I populations, we speculate that the selection could be associated with the emergence of agriculture or other culture shifts that increased the efficiency of food procurement. The reduced glucose tolerance associated with the derived GIP haplotype may provide a protective endocrine response under challenging environments. For example, an elevated glucose level during pregnancy may improve the survival of fetuses under extreme malnutrition conditions, despite that an impaired glucose tolerance response represents a risk to both the mother and fetus under normal conditions. Therefore, the derived GIP haplotype may be advantageous if the population experienced frequent bouts of famine, a phenomenon commonly found in agriculture societies.

The relationship between GIP haplotypes and the glucose challenge response was uncovered by studying polymorphisms exhibiting moderate degree of population differentiation. This observation may help explain how the selection of GIP haplotypes evaded identification in earlier studies of positive selection in humans. It is conceivable that loci where genes were subjected to temporally heterogeneous selection were routinely excluded in searches of signatures of positive selection using stringent statistical thresholds. Therefore, not only are novel markers provided for stratification for the risk of aberrant glucose metabolism in patients but also an example for exploring metabolism modifiers that were partially selected or were in balance selection.

### Materials and Methods

Patients. The study was approved by the institutional ethics committee review boards of the Chang Gung Memorial Hospital Linkou Medical Center and the Chang Gung University. All patients gave written informed consent to participate in the study. Age of patients ranged from 19-42 (30.9±0.4) years, and the body mass index [weight (kg) divided by height squared (m)] (BMI) was 25.7±0.4 kg/m².

To perform the screening glucose challenge test for gestational diabetes, patients during the 23rd to the 29th week of pregnancy were given a sweetened liquid (Glucola, which contains 50 g of glucose), and were instructed to drink quickly. A trained nurse collected study data by interviewing patients, reviewing their medical records, supervising the glucose intake, and obtaining a venous blood sample about 60 minutes after drinking the glucose solution for DNA extraction and glucose measurement. For glucose challenge response analysis, a 140 mg/dL threshold that allows the identification of 80% of women with gestational diabetes was used as the cutoff value for statistical analysis.

Genotyping. Genomic DNA samples of subjects were extracted and purified from anticoagulated blood by the DNeasy Blood & Tissue Kit (Qiagen). Genotyping of SNPs was performed using the Applied Biosystems TaqMan® Validated SNP Genotyping Assays with a 7500 Real-Time PCR System (Stanford University Protein and Nucleic Acid Biotechnology Facility). Six SNPs were analyzed for all patients. These SNPs included rs3895874, rs3809770, rs3848460, and rs937301 at the proximate region of GIP promoter as well as the intronic rs10423928 and missense rs1800437 in GIPR, which encodes the cognate receptor for GIP. These linked SNPs in GIPR are referred to as GIPR^{1159C/G} mutation in the Table 5. The run and analysis were performed using the StepOne™ Software v2.1. Because GIP variants at the 5′ gene region were partially selected in East Asian, we chose the homogenous, but unrelated, Han-Chinese patients for testing. By choosing unrelated individuals, it is unlikely that the association we found is due to stratification of samples.

FST estimation and LD plot. We computed and presented the unbiased estimate of the population genetic differentiation statistic FST in the HapMap project phase I+II+III using the PGEToolbox and the SPSmart v4. The FST test is robust at detecting rises in allele frequency and highly differentiated alleles among populations. SNPs with an FST value above the 5% cutoff were further analyzed for the presence of linkage disequilibrium (LD) within neighboring gene regions. LD and haplotype plots for SNPs within the neighboring region of the GIP locus were generated using the HaploView 4.1. Default settings were used, and 95% confidence intervals for the disequilibrium coefficient (D′) were calculated to identify pairwise SNPs in strong LD.

In Vitro GIP promoter reporter analysis. A 2.15 kb fragment of human GIP promoter with the A allele at rs3895874, rs3809770, rs3848460, and rs937301 (−2073 bp to +77 bp; referred to as GIP−1920AAAA haplotype) was chemically synthesized (Genescript Inc.), and subcloned into the pGL4.2 luciferase reporter vector (Promega Corp.). The identity of promoter sequence was verified by direct sequencing. To obtain promoter fragment with ancestral haplotypes that differ at rs3809770 (referred to as GIP^{-1920GAGG} and GIP^{-1920GGGG} haplotypes), site-directed mutagenesis was performed. The PAX6 and GATA4 expression vectors (subcloned in the pCMV6-XL5 and pCMV6-XL4 vector, respectively) were obtained from OriGene Technologies, Inc. To compare GIP promoter reporter activities, HEK293T cells in 6-well culture dishes were transfected with different combinations of transcription factor expression vector and a select GIP promoter reporter using lipofectamine 2000. At 48 hr after transfection, cells were free-thawed once in lysis buffer, and luciferase activity in supernatant was assayed using the Steady-Glo® Luciferase Assay System (Promega Corp.) and a Lumimark microplate reader (BioRad Corp.). In all analyses, cells were co-transfected with a pCMV-β-galactosidase vector to monitor transfection efficiency. The reporter activity is expressed as the ratio of relative luciferase unit/β-galactosidase activity in transfected cells.

Hitchhiking mapping for GIP locus and neighboring regions. A window of 20 SNPs (with the step of one SNP) was used to scan for signatures of selective sweeps within the 2 Mbp regions around the focal gene GIP (HapMap I+II dataset). For each window, we excluded coding SNPs and computed values of heterozygosity and Fay & Wu's H from all noncoding SNPs in YRI, CEU, and ASN populations. Extended haplotype homozygosity (EHH) analyses were performed using the HapMap dataset as described earlier. The heterozygosity for a window was calculated as the arithmetic average of heterozygosities of the SNPs in that window. The estimator for heterozygosity of the SNP was 2p₁(1−p₁)[n/(n−1)], where n is the sample size, and pi the allele frequency of the SNP. The D and H statistics were computed using PGEToolbox. To compute H, we determined the ancestral state for each SNP by using the alignments between human and chimpanzee genome sequences: if an allele of a human SNP is identical to the base of aligned chimpanzee sequence, the allele was regarded as the ancestral allele. Some SNPs whose ancestral states could not be determined (e.g., either allele of a human SNP differs from the base of chimpanzee sequence, or the chimpanzee base is undetermined) were excluded. Values of H were normalized and validated with an independent implement.

Statistical Analysis. All results of SNP genotyping were screened for deviations from Hardy-Weinberg equilibrium; no SNPs showed significant deviation. The glucose challenge responses among patients were compared with the chi-square test and the Student's t-test with Welch's correction. All P-values were two-sided. All data were presented as mean±SEM.

1. An isolated polypeptide comprising the amino acid sequence set forth as: YAEGTFISDYSIAMDKIHQQDFVNWLLAQKGKKNDWKHNITQREARALELA S/G QAN; and
functional fragments, derivatives and homologs thereof.

2. An isolated polypeptide according to claim 1, wherein said polypeptide is GIP55S.

3. An isolated polypeptide according to claim 1, wherein said polypeptide is GIP55G.

4. An isolated polypeptide comprising at least 40 contiguous amino acids of the polypeptide according to claim 1.

5. A pharmaceutical composition comprising: a therapeutically effective amount of a GIP peptide as set forth in claim 1; and a pharmaceutically acceptable carrier.

6. An antibody that specifically recognizes a GIP peptide as set forth in claim 1.

7. A method of treating or preventing the onset of diabetes in an individual, the method comprising:
administering to said individual an effective dose of a GIP peptide as set forth in claim 1.

8. The method of claim 7, further comprising administering an effective dose of GLP-1, or a pharmaceutically active analog thereof.

9. A method of diagnosing altered GIP physiology in an individual, comprising determining the presence or absence of at least one polymorphic allele in a biological sample from said individual, wherein the at least one polymorphic allele is selected from the polymorphisms 36-79 of Table 2.

10. The method of claim 9, wherein the altered GIP physiology results in type II diabetes or obesity.

11. The method of claim 9, wherein said biological sample is a genetic sample.

12. The method of claim 11, wherein the genetic sample comprises mRNA or a cDNA derived therefrom.

13. The method of claim 10, wherein the polymorphic allele differs in the GIP promoter region.

14. The method of claim 13, wherein the polymorphic allele corresponds to the GIP^{-1920A/A} genotype.

15. A kit for assessing altered GIP physiology in an individual, the kit comprising reagents for selectively determining the presence or absence of at least one polymorphic allele in a biological sample from said individual, wherein the at least one polymorphic allele is one of polymorphisms 36-79 of Table 2.

16. The kit according to claim 13, comprising probes that specifically bind to at least one of polymorphisms 36-79 of Table 2.


SEQUENCE LISTING (116 sequences)

SEQ ID NO: 1 - protein, 55 aa, H. sapiens
YAEGTFISDYSIAMDKIHQQDFVNWLLAQKGKKNDWKHNITQREARALELASQAN

SEQ ID NO: 2 - protein, 55 aa, H. sapiens
YAEGTFISDYSIAMDKIHQQDFVNWLLAQKGKKNDWKHNITQREARALELAGQAN

SEQ ID NO: 3 - protein, 57 aa, H. sapiens
YAEGTFISDYSIAMDKIHQQDFVNWLLAQKGKKNDWKHNITQREARALELASQANRK

SEQ ID NO: 4 - protein, 42 aa, H. sapiens
YAEGTFISDYSIAMDKIHQQDFVNWLLAQKGKKNDWKHNITQ

SEQ ID NO: 5 - protein, 57 aa, Pan troglodytes; identical to SEQ ID NO: 3

SEQ ID NO: 6 - protein, 57 aa, Pongo pygmaeus
YAEGTFISDYSIAMDKIHQQDFVNWLLAQKGKKNEWKHNITQREARALELASQANRK

SEQ ID NO: 7 - protein, 57 aa, Gorilla gorilla
YAEGTFISDYSIAMDKIHQQDFVNWLLAQKGKKNEWKHNITQREAQALELASQANRK

SEQ ID NO: 8 - protein, 57 aa, Macaca mulatta; identical to SEQ ID NO: 3

SEQ ID NO: 9 - protein, 57 aa, Canis lupus familiaris
YAEGTFISDYSIAMDKIRQQDFVNWLLAQKGKKNDWKHNITQREAGALELAHQSNRK

SEQ ID NO: 10 - protein, 57 aa, Bos primigenius
YAEGTFISDYSIAMDKIRQQDFVNWLLAQKGKKSDWIHNITQREAGALELAHQSNRK

SEQ ID NO: 11 - protein, 57 aa, Felis catus
YAEGTFISDYSIAMDKIRQQDFVNWLLAQKGKKNEWKHNITQREAGALELAHQSNRK

SEQ ID NO: 12 - protein, 57 aa, Sus domestica
YAEGTFISDYSIAMDKIRQQDFVNWLLAQKGKKSEWKHNITQREARALELAHQSNRK

SEQ ID NO: 13 - protein, 57 aa, Equus ferus caballus
YAEGTFISDYSIAMDKIRQQDFVNWLLAQKGKKNDWKHNITQREARALELTHQSNRK

SEQ ID NO: 14 - protein, 57 aa, Rattus rattus
YAEGTFISDYSIAMDKIRQQDFVNWLLAQKGKKNDWKHNLTQREARALELAGQSQRN

SEQ ID NO: 15 - protein, 57 aa, Mus musculus
YAEGTFISDYSIAMDKIRQQDFVNWLLAQRGKKSDWKHNITQREARALVLAGQSQGK

SEQ ID NO: 16 - protein, 57 aa, Cavia porcellus
YAEGTFISDYSIAMDKIRQQDFVNWLLAQKGKKGDWSHNLTQRDTHNPELSRPSQER

SEQ ID NO: 17 - protein, 57 aa, Loxodonta africana
YAEGTFISDYSIAMDKIRQQDFVNWLLAQKGKKNEWKHNITQREARALELAVQSNRN

SEQ ID NO: 18 - protein, 59 aa, Didelphis marsupialis
YAEGTFISDYSITMDKIMQQDFVNWLLSQKGKKNSWRHNITERKADGTGLTNHSNWNLK

SEQ ID NO: 19 - protein, 60 aa, Gallus gallus domesticus
YSEATLASDYSRTMDNMLKKNFVEWLLARREKKSDNVIEPYKREAEPQLSAVSDQSLDPR

SEQ ID NO: 20 - protein, 57 aa, Xenopus laevis
YSEAILASDYSRSVDNMLKKNFVDWLLARREKKSENTSEATKREADFQLPDVNMKEK

SEQ ID NO: 21 - protein, 53 aa, Danio rerio
YAESTIASDISKIVDSMVQKNFVNFLLNQREKKSEPALTEDPESHIFNDLLKK

SEQ ID NO: 22 - DNA, 53 bp, H. sapiens
caggaggaaggggacgggtaggcacgaacagacggtatatgaacgggcaaatg

SEQ ID NO: 23 - DNA, 53 bp, H. sapiens
gtaaagctgatcaagggacacatgtctataaataccgtacgggcaggtagatg

SEQ ID NO: 24 - DNA, 53 bp, H. sapiens
gtgaaggaaggcaaggggtacgcacgaacagacgctgtatgagcgggcagata

SEQ ID NO: 25 - DNA, 53 bp, H. sapiens
gtgggagaaggcgggaggtacgcacgaacaggcgccgcgcaggtaactggggg

SEQ ID NO: 26 - DNA, 49 bp, H. sapiens
aggtgatcaaggggtacgcacgtacagatggtgtacgggcaggcagatg

SEQ ID NO: 27 - DNA, 27 bp, H. sapiens
aggagatcaaggtgtacgcacgaacgg

SEQ ID NO: 28 - DNA, 18 bp, H. sapiens
agggacacatgtgtacaa

SEQ ID NO: 29 - DNA, 33 bp, H. sapiens
gtatctctaatcggcactcaggggttggcaccg

SEQ ID NO: 30 - DNA, 33 bp, H. sapiens
acacctctaatcggcattcgggagcttatggcg

SEQ ID NO: 31 - DNA, 33 bp, H. sapiens
gcacctctaatcggcattcgaaaacctgcgccg

SEQ ID NO: 32 - DNA, 26 bp, H. sapiens
gcgcgctcgcctactctcttgcgctg

SEQ ID NO: 33 - DNA, 26 bp, H. sapiens
ggacctctaatcggcattttgtggcg

SEQ ID NO: 34 - DNA, 26 bp, H. sapiens
gcatctctaatcggcattcggcacca

SEQ ID NO: 35 - skipped

SEQ ID NO: 36 - DNA, 72 bp, H. sapiens
caggaggaaggggacgggtaggcacgaaaggacgggtatatgaacgggcaaatggtatctctaatcggcact

SEQ ID NO: 37 - DNA, 72 bp, H. sapiens
gtaaaggaaggcaaggtgtgcgcacgagggcacgcacgcgcaggtaactggggggcgcgctcgcctactctc

SEQ ID NO: 38 - DNA, 72 bp, H. sapiens
gtgaagctgatcaagggacacatgtctaaacgcgcacgcgcaggtaactggggggcgcgctcgcctactctc

SEQ ID NO: 39 - DNA, 69 bp, H. sapiens
cagagagaaggcgggaggtacgcacgaaacgcacgtacgagcgggcagatagcacctctaatcggcatt

SEQ ID NO: 40 - DNA, 41 bp, H. sapiens
atacacgtacgggcaggtagatgacacctctaatcggcatt

SEQ ID NO: 41 - DNA, 41 bp, H. sapiens
acgggtgtatgaacgggcaaatggtatctctaatcggcact

SEQ ID NO: 42 - DNA, 20 bp, H. sapiens
caggggcttatgcgtagcta

SEQ ID NO: 43 - DNA, 20 bp, H. sapiens
tgaaaattggcacgggcccc

SEQ ID NO: 44 - DNA, 20 bp, H. sapiens
cgggagcctgcgcaggcgcc

SEQ ID NO: 45 - DNA, 20 bp, H. sapiens
cgggggcttgcgcgtagccc

SEQ ID NO: 46 - DNA, 8 bp, H. sapiens
cgtagcca

SEQ ID NO: 47 - DNA, 35 bp, H. sapiens
cagggagaaggcgggaggtacgcacgaacagggcg

SEQ ID NO: 48 - DNA, 35 bp, H. sapiens
gtgaaggaaggcaaggtgtgcgcacgagcggcacg

SEQ ID NO: 49 - DNA, 35 bp, H. sapiens
gtagaggaaggggacgggtaggcacgaacaggacg

SEQ ID NO: 50 - DNA, 32 bp, H. sapiens
aagctgatcaagggacacatgtctataacata

SEQ ID NO: 51 - DNA, 49 bp, H. sapiens
cacgcgcaggtaactggggggcgcgctcgcctactctctgaaaatgcgc

SEQ ID NO: 52 - DNA, 49 bp, H. sapiens
cacgtacgagcgggcagatagcacctctaatcggcattcgggggtatgg

SEQ ID NO: 53 - DNA, 49 bp, H. sapiens
ggtatatgaacgggcaaatggtatctctaatcggcactcaggggggcac

SEQ ID NO: 54 - DNA, 49 bp, H. sapiens
cacgtacgggcaggtagatgacacctctaatcggcattcgggagtgcac

SEQ ID NO: 55 - DNA, 5 bp, H. sapiens
tgtgg

SEQ ID NO: 56 - DNA, 37 bp, H. sapiens
cgcgcaggtaactggggggcgcgctcgcctactctct

SEQ ID NO: 57 - DNA, 37 bp, H. sapiens
tatatgaacgggcaaatggtatctctaatcggcactc

SEQ ID NO: 58 - DNA, 37 bp, H. sapiens
tgtatgaacgggcaaatggtatctctaatcggcactt

SEQ ID NO: 59 - DNA, 37 bp, H. sapiens
cgtacgagcgggcagatagcacctctaatcggcattc

SEQ ID NO: 60 - DNA, 37 bp, H. sapiens
tgtatgagcgggcagatagcacctctaatcggcattc

SEQ ID NO: 61 - DNA, 37 bp, H. sapiens
cgtacgagcgggcagatagcatctctaatcggcattt

SEQ ID NO: 62 - DNA, 37 bp, H. sapiens
tgtatgagcgggcagatagcatctctaatcggcattt

SEQ ID NO: 63 - DNA, 37 bp, H. sapiens
cgtacgggcaggtagatgacacctctaatcggcattc

SEQ ID NO: 64 - DNA, 37 bp, H. sapiens
tgtacgggcaggtagatgacacctctaatcggcattc

SEQ ID NO: 65 - DNA, 37 bp, H. sapiens
cgtacgggcaggcagatggcacctctaatcggcattc

SEQ ID NO: 66 - DNA, 37 bp, H. sapiens
cgtacgggcaggcagatggcacctctaatcggcattt

SEQ ID NO: 67 - DNA, 37 bp, H. sapiens
tgtatgggcaggcagatggcacctctaatcggcattc

SEQ ID NO: 68 - DNA, 37 bp, H. sapiens
tgtatgggcaggcagatggcacctctaatcggcattt

SEQ ID NO: 69 - DNA, 36 bp, H. sapiens
cgcgcgggcaagtaggggcacccccgaccggcattc

SEQ ID NO: 70 - DNA, 35 bp, H. sapiens
caggaggaaggggacgggtaggcacgaacagacgg

SEQ ID NO: 71 - DNA, 35 bp, H. sapiens
gtaaagctgatcaagggacacatgtctataaatac

SEQ ID NO: 72 - DNA, 35 bp, H. sapiens
gtgaaggaaggcaaggggtacgcacgaacagacgc

SEQ ID NO: 73 - DNA, 35 bp, H. sapiens
gtgggagaaggcgggaggtacgcacgaacaggcgc

SEQ ID NO: 74 - DNA, 31 bp, H. sapiens
aggtgatcaaggggtacgcacgtacagatgg

SEQ ID NO: 75 - DNA, 27 bp, H. sapiens
aggagatcaaggtgtacgcacgaacgg

SEQ ID NO: 76 - DNA, 18 bp, H. sapiens
aggtgtgcgcacgagcgg

SEQ ID NO: 77 - DNA, 18 bp, H. sapiens
agggacacatgtgtacaa

SEQ ID NO: 78 - DNA, 28 bp, H. sapiens
caggaggaaggggacgggtaggcacgaa

SEQ ID NO: 79 - DNA, 28 bp, H. sapiens
gtaaaggaaggcaaggtgtgcgcacgag

SEQ ID NO: 80 - DNA, 28 bp, H. sapiens
gtgaagctgatcaagggacacatgtcta

SEQ ID NO: 81 - DNA, 28 bp, H. sapiens
gagagagaaggcgggaggtacgcacgaa

SEQ ID NO: 82 - DNA, 30 bp, H. sapiens
cagggagaaggcgggaggtacgcacgaaca

SEQ ID NO: 83 - DNA, 30 bp, H. sapiens
gtgaaggaaggcaaggtgtgcgcacgagcg

SEQ ID NO: 84 - DNA, 30 bp, H. sapiens
gtagaggaaggggacgggtaggcacgaaca

SEQ ID NO: 85 - DNA, 27 bp, H. sapiens
aagctgatcaagggacacatgtctata

SEQ ID NO: 86 - DNA, 37 bp, H. sapiens; identical to SEQ ID NO: 57

SEQ ID NO: 87 - DNA, 37 bp, H. sapiens; identical to SEQ ID NO: 63

SEQ ID NO: 88 - DNA, 37 bp, H. sapiens; identical to SEQ ID NO: 60

SEQ ID NO: 89 - DNA, 37 bp, H. sapiens; identical to SEQ ID NO: 56

SEQ ID NO: 90 - DNA, 37 bp, H. sapiens
tgtacgggcaggcagatgggacctctaatcggcattt

SEQ ID NO: 91 - DNA, 19 bp, H. sapiens
gcatctctaatcggcattc

SEQ ID NO: 92 - DNA, 44 bp, H. sapiens
aggacgggtatatgaacgggcaaatggtatctctaatcggcact

SEQ ID NO: 93 - DNA, 44 bp, H. sapiens
ggcacgcacgcgcaggtaactggggggcgcgctcgcctactctc

SEQ ID NO: 94 - DNA, 44 bp, H. sapiens
aacgcgcacgcgcaggtaactggggggcgcgctcgcctactctc

SEQ ID NO: 95 - DNA, 41 bp, H. sapiens
acgcacgtacgagcgggcagatagcacctctaatcggcatt

SEQ ID NO: 96 - DNA, 41 bp, H. sapiens; identical to SEQ ID NO: 40

SEQ ID NO: 97 - DNA, 41 bp, H. sapiens; identical to SEQ ID NO: 41

SEQ ID NO: 98 - DNA, 43 bp, H. sapiens
gggcgcacgcgcaggtaactggggggcgcgctcgcctactctc

SEQ ID NO: 99 - DNA, 43 bp, H. sapiens
gcacgcacgtacgagcgggcagatagcacctctaatcggcatt

SEQ ID NO: 100 - DNA, 43 bp, H. sapiens
ggacgggtatatgaacgggcaaatggtatctctaatcggcact

SEQ ID NO: 101 - DNA, 43 bp, H. sapiens
acatacacgtacgggcaggtagatgacacctctaatcggcatt

SEQ ID NO: 102 - DNA, 14 bp, H. sapiens
aggggttggcaccg

SEQ ID NO: 103 - DNA, 14 bp, H. sapiens
gggagcttatggcg

SEQ ID NO: 104 - DNA, 14 bp, H. sapiens
gaaaacctgcgccg

SEQ ID NO: 105 - DNA, 7 bp, H. sapiens
tgcgctg

SEQ ID NO: 106 - DNA, 7 bp, H. sapiens
tgtggcg

SEQ ID NO: 107 - DNA, 7 bp, H. sapiens
ggcacca

SEQ ID NO: 108 - DNA, 12 bp, H. sapiens
caggggcttatg

SEQ ID NO: 109 - DNA, 12 bp, H. sapiens
tgaaaattggca

SEQ ID NO: 110 - DNA, 12 bp, H. sapiens
cgggagcctgcg

SEQ ID NO: 111 - DNA, 12 bp, H. sapiens
cgggggcttgcg

SEQ ID NO: 112 - DNA, 11 bp, H. sapiens
tgaaaatgcgc

SEQ ID NO: 113 - DNA, 11 bp, H. sapiens
cgggggtatgg

SEQ ID NO: 114 - DNA, 11 bp, H. sapiens
caggggggcac

SEQ ID NO: 115 - DNA, 11 bp, H. sapiens
cgggagtgcac

SEQ ID NO: 116 - DNA, 5 bp, H. sapiens
tgtgg